(19) United States
(12) Patent Application Publication
(10) Pub. No.: US 2024/0382472 A1
(43) Pub. Date: Nov. 21, 2024

(54) NEUTROPHIL ELASTASE INHIBITORS FOR USE IN THE TREATMENT OF FIBROSIS

(71) Applicants: MEREO BIOPHARMA 4 LIMITED, Greater London (GB); THE UNITED STATE OF AMERICA AS REPRESENTED BY THE SECRETARY, DEPARTMENT OF HEALTH AND HUMAN SERVICES, Bethesda, MD (US)

(72) Inventors: Jacqueline PARKIN, Greater London (GB); Steven Z. PAVLETIC, Bethesda, MD (US); Annie IM, Pittsburgh, PA (US); Noa G. HOLTZMAN, Bethesda, MD (US); Cody J. PEER, Bethesda, MD (US)

(73) Assignees: MEREO BIOPHARMA 4 LIMITED, Greater London (GB); THE UNITED STATE OF AMERICA AS REPRESENTED BY THE SECRETARY, DEPARTMENT OF HEALTH AND HUMAN SERVICE, Bethesda, MD (US)

(21) Appl. No.: 18/692,523

(22) PCT Filed: Oct. 20, 2022
(86) PCT No.: PCT/EP2022/079286

§ 371 Date: Mar. 15, 2024

Related U.S. Application Data
(60) Provisional application No. 63/262,788, filed on Oct. 20, 2021.

(51) Int. Cl. A61K 31/444; A61P 11/00; A61P 37/06

(52) U.S. Cl. CPC A61K 31/444; A61P 11/00; A61P 37/06

(57) ABSTRACT

The invention relates to treatments for fibrosis by administering a neutrophil elastase inhibitor, such as alvelestat. In particular, the invention relates to treatments for fibrosis in combination with another disease, such as organ rejection, for example bronchiolitis obliterans syndrome optionally associated with graft-versus-host-disease.

### CROSS REFERENCE TO RELATED APPLICATIONS

This application claims the benefit of priority of US provisional application U.S. 63/262,788, filed 20 Oct. 2021. The contents of this application are incorporated herein by reference.

### FIELD OF THE INVENTION

The present invention relates to new methods for treating or preventing fibrosis, comprising administering a neutrophil elastase inhibitor, in particular alvelestat or a pharmaceutically acceptable salt and/or solvate thereof, to a subject in need thereof.

### BACKGROUND TO THE INVENTION

### Fibrosis

Fibrosis (also known as fibrotic scarring) is a leading cause of morbidity and/or mortality and has no treatment. Fibrosis refers to the development of fibrous connective tissue as a reparative response to injury or damage and also to the deposition of excess fibrous connective tissue that occurs as a pathological process. Fibrous connective tissue comprises collagen fibers. Fibrosis can occur in many tissues within the body and can arise from inflammation, injury and/or tissue (e.g. organ) transplant. Examples of tissues include the lungs, liver, brain, heart, gastrointestinal tract, and skin.

There is a link between the synthesis of collagen, a major component of the extracellular matrix (ECM), and fibrosis.[1] In particular, in the ECM there is an equilibrium in the production, deposition and remodelling of collagen. However, during fibrosis this equilibrium is disrupted and results in collagen being deposited. An increase in the accumulation of ECM can also contribute to the pathogenesis of fibrosis. In addition, tissues affected by inflammation can be susceptible to fibrosis. This is because inflammation can cause an excessive accumulation of extracellular matrix (ECM) components, such as collagen proteins, which leads to fibrosis that ultimately interferes with normal tissue function.

### Fibrosis and Graft Rejection

Fibrosis can be observed following the transplant of organs. Transplant of organs, bone marrow and human stem cells has advanced human health. However, transplantation is beset by the immune system's ability to recognise and react to non-self tissue. This is particularly a risk in allogeneic transplants when the tissue is from genetically similar, but not identical donors and there is a human leukocyte antigen (HLA) tissue type mismatch.

Graft rejection following solid organ transplantation can occur when the recipient's immune system (in particular the recipient's mature αβ T cells) recognises the foreign HLA antigens expressed on the donor organ's cells. It is dictated by host allo-responsiveness against mismatched donor antigens. Acute rejection typically occurs within the first weeks to several months after transplantation and is a major risk factor for the development of chronic rejection. Other risk factors for chronic rejection include inflammation and/or infection. Chronic rejection typically develops within months to years after transplantation and is the major cause of long-term graft loss. Clinically, chronic rejection can involve the replacement of the allograft parenchyma with fibrous scar tissue (i.e. fibrosis). Hence fibrosis associated with graft rejection is undesirable.

### Fibrosis and Transplantation

Lung transplantation is an important treatment option for patients with advanced lung disease or irreversible pulmonary failure: around 3,500 lung transplantations are performed globally each year. However, acute lung rejection affects about a third of all lung transplant recipients within the first year after transplant, and has an increased risk of developing into chronic lung rejection (or chronic lung allograft dysfunction (CLAD)) which remains a major hurdle to long-term survival post lung transplantation. It is the leading cause of allograft loss and death for recipients of lung transplants surviving beyond 3 months post-transplant.

Lung Transplant associated Bronchiolitis Obliterans Syndrome (LT-BOS) is a form of CLAD, and manifests as a decline of lung function, which is often progressive. It is thought to be caused by inflammation, destruction and/or fibrosis of small airways in the lung allograft that leads to obliterative bronchiolitis (OB). Median survival after diagnosis is between 3-5 years [2]. Lung Transplant associated Restrictive Allograft Syndrome (RAS) is another form of CLAD which can manifest as a chronic, persistent restrictive decline in pulmonary function.[3] RAS exhibits characteristics of peripheral lung fibrosis and significantly affects survival of lung transplant patients. Hence, fibrosis associated with graft rejection is undesirable.

Despite the high incidence, there is currently no adequate treatment for chronic graft rejection, in particular for LT-BOS. Current options for chronic graft rejection, in particular LT-BOS, include immunosuppression therapy (often a triple combination), and neo-macrolides (such as azithromycin), as well as treatment of accompanying gastro-oesophageal reflux and infections. However, the evidence to support currently available therapies is limited, therapeutic response is typically poor, and the risk of serious adverse events is high: immunosuppression therapy greatly impairs immune reconstitution, which increases the risk of infections. As a last resort, lung re-transplantation may be considered, but outcomes are poor and donor organs are scarce. As a result, the ISHLT/ATS/ERS BOS Task Force concluded in 2014 that no currently available therapies have been proven to result in significant benefit in the prevention or treatment of LT-BOS [4].

### Fibrosis and Graft Versus Host Disease

As a further complication in transplant rejection, an immune response mounted against the recipient of an allograft by mature donor αβ T cells contained in the graft can lead to graft versus host disease (GVHD). Typically, GVHD is seen in the context of allogeneic haematopoietic stem-cell transplantation, although it can also occur in immunodeficient patients when they receive blood transfusions. Acute GVHD is characterized by damage to the skin, liver, lung and/or the gastrointestinal tract, whereas chronic GVHD has more diverse manifestations and can resemble autoimmune syndromes. Standard of care is immunosuppression therapy, but as discussed above this carries a high risk of adverse events and increases the risk of infections [5]. Following stem cell transplantation, a subject can develop GVHD BOS, where BOS is an example of an obstructive lung disease. Alternatively, following stem cell transplantation, a subject can develop restrictive lung diseases such as GVHD associated restrictive chronic lung function decline (GVHD R-CLFD).[6] Restrictive and obstructive lung diseases can include a fibrotic aspect. Hence, fibrosis associated with GVHD is undesirable.

### Neutrophil Elastase (NE) Inhibitors

NE inhibitors have been implicated in the treatment of various diseases. However, the mechanism connecting neutrophil elastase inhibition and reducing fibrosis—in particular in fibrosis associated with GVHD—is poorly understood and not well established. Furthermore, it is believed that a therapeutic effect in humans had not previously been demonstrated.

Alvelestat has been investigated in the treatment chronic graft rejection (in particular LT-BOS) and GVHD (see WO2021/053058 herein incorporated by reference) and was shown to improve lung function. However, it is not disclosed there that inhibition of NE using alvelestat would specifically be useful in treating the fibrosis associated with graft rejection or GVHD, or fibrosis associated with other diseases.

There is therefore a need for new therapies for treating and preventing fibrosis. In particular, there is a need for new therapies for treating and preventing fibrosis associated with diseases selected from chronic graft rejection (in particular LT-BOS) and GVHD (in particular GVHD BOS). In addition, there a need for new therapies for treating and preventing diseases comprising reducing fibrosis.

### SUMMARY OF THE INVENTION

It is demonstrated that, surprisingly, inhibitors of neutrophil elastase (NE) such as alvelestat are useful in the treatment and prevention of fibrosis, in particular fibrosis associated with GVHD BOS, in particular in a human subject.

The invention described herein is unexpected because to the best of our knowledge there is no established link between the mechanisms of fibrosis and NE. For example, a main driver of fibrosis is typically considered to be excess accumulation and/or deposition of extracellular matrix (ECM) components such as collagen, rather than neutrophils. NE inhibitors have not previously been demonstrated to be effective in treating or preventing fibrosis associated with BOS or GVHD in a subject. Indeed, to the best of our knowledge NE inhibition has not previously been demonstrated for treatment of fibrosis in a human subject in need thereof.

Thus the present invention provides a method for treating or preventing fibrosis, comprising administering an effective amount of a neutrophil elastase inhibitor, in particular alvelestat or a pharmaceutically acceptable salt and/or solvate thereof, to a subject in need thereof.

The present invention further provides a method for treating or preventing fibrosis associated with a tissue, comprising administering an effective amount of a neutrophil elastase inhibitor, in particular alvelestat or a pharmaceutically acceptable salt and/or solvate thereof, to a subject in need thereof.

The present invention further provides a method for treating or preventing fibrosis associated with a disease, comprising administering an effective amount of a neutrophil elastase inhibitor, in particular alvelestat or a pharmaceutically acceptable salt and/or solvate thereof, to a subject in need thereof.

The present invention further provides a method for treating or preventing fibrosis associated with graft rejection, comprising administering an effective amount of a neutrophil elastase inhibitor, in particular alvelestat or a pharmaceutically acceptable salt and/or solvate thereof, to a subject in need thereof.

The present invention further provides a method for treating or preventing graft rejection, comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject.

The present invention further provides a method for treating or preventing fibrosis associated with lung transplant associated bronchiolitis obliterans syndrome (LT-BOS), comprising administering an effective amount of a neutrophil elastase inhibitor, in particular alvelestat or a pharmaceutically acceptable salt and/or solvate thereof, to a subject in need thereof.

The present invention further provides a method for treating or preventing lung transplant associated bronchiolitis obliterans syndrome (LT-BOS), comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject.

The present invention further provides a method for treating or preventing fibrosis associated with lung transplant associated Restrictive Allograft Syndrome (LT-RAS), comprising administering an effective amount of a neutrophil elastase inhibitor, in particular alvelestat or a pharmaceutically acceptable salt and/or solvate thereof, to a subject in need thereof.

The present invention further provides a method for treating or preventing lung transplant associated Restrictive Allograft Syndrome (LT-RAS), comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject.

The present invention further provides a method for treating or preventing fibrosis associated with graft versus host disease (GVHD), comprising administering an effective amount of a neutrophil elastase inhibitor, in particular alvelestat or a pharmaceutically acceptable salt and/or solvate thereof, to a subject in need thereof.

The present invention further provides a method for treating or preventing graft versus host disease (GVHD), comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject.

The present invention further provides a method for treating or preventing fibrosis associated with GVHD BOS, comprising administering an effective amount of a neutrophil elastase inhibitor, in particular alvelestat or a pharmaceutically acceptable salt and/or solvate thereof, to a subject in need thereof.

The present invention further provides a method for treating or preventing GVHD BOS, comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject. In some embodiments, the subject has received a hematopoietic stem cell transplant.

The present invention further provides a method for treating or preventing fibrosis associated with GVHD R-CLFD, comprising administering an effective amount of a neutrophil elastase inhibitor, in particular alvelestat or a pharmaceutically acceptable salt and/or solvate thereof, to a subject in need thereof.

The present invention further provides a method for treating or preventing GVHD R-CLFD, comprising administering an effective amount of a neutrophil elastase inhibitor, in particular alvelestat or a pharmaceutically acceptable salt and/or solvate thereof, to a subject in need thereof, wherein the treatment comprises reducing fibrosis in the subject. In some embodiments, the subject has received a hematopoietic stem cell transplant.

In the methods of the invention described herein, the subject is preferably a human subject.

In addition, as shown in the Examples, analysis of biomarkers such as desmosine and isodesmosine (DES and IDES or DES/IDES) indicate that NE activity is raised in GVHD BOS. This observation may facilitate the use of biomarkers to assist in the determination of NE levels and the identification of subjects in need of NE inhibition.

Thus, the present invention provides a method of identifying a subject in need of treatment with alvelestat or a pharmaceutically acceptable salt thereof, comprising determining the level of desmosine and isodesmosine in a sample from a subject, wherein a raised level of desmosine and isodesmosine relative to a baseline or a reference level identifies the subject in need of treatment.

The present invention also provides a method of determining neutrophil elastase activity, comprising evaluating the level of desmosine and isodesmosine in a sample from a subject, wherein a raised level of desmosine and isodesmosine relative to a baseline or a reference level indicates raised neutrophil elastase activity.

In particular, the present invention provides a method of identifying a subject in need of treatment with an NE inhibitor, in particular alvelestat, comprising the steps of:
obtaining a sample from a subject,
measuring the level of desmosine and isodesmosine in the sample, and comparing the level against a baseline or a reference level,
wherein a raised level of desmosine and isodesmosine relative to the baseline or the reference level indicates raised neutrophil elastase activity and that the subject is in need of treatment with an NE inhibitor, in particular alvelestat.

In particular, the present invention provides a method of determining neutrophil elastase activity comprising the steps of:
obtaining a sample from a subject,
measuring the level of desmosine and isodesmosine in the sample, and comparing the level against a baseline or a reference level,
wherein a raised level of desmosine and isodesmosine relative to the baseline or the reference level indicates raised neutrophil elastase activity.

In addition, as shown in the Examples, the analysis of a ratio of two biomarkers, such as a biomarker of collagen synthesis (e.g. PRO-C3) to a biomarker of collagen degradation (e.g. C3M) serve as indicators fibrotic activity in subject with GVHD BOS. This observation may facilitate the use of biomarker ratios to assess fibrotic activity.

Thus, the present invention also provides a method of monitoring fibrotic activity in a patient undergoing treatment with a neutrophil elastase inhibitor, in particular alvelestat, the method comprising the steps of:
obtaining a sample from a subject,
measuring the level of a biomarker of collagen synthesis (e.g. PRO-C3) in the sample and measuring the level of a biomarker of collagen degradation (e.g. C3M) in the sample,
evaluating the ratio of the level of the biomarker of collagen synthesis (e.g. PRO-C3) to the level of the biomarker of collagen degradation (e.g. C3M) in a subject,
comparing the ratio against a baseline or a reference ratio,
wherein a decrease in the ratio relative to the baseline or the reference ratio indicates a decrease in fibrotic activity.

Thus, the present invention also provides a method of identifying a subject in need of treatment with a neutrophil elastase inhibitor, in particular alvelestat, wherein the subject has a fibrotic disease, the method comprising the steps of:
-obtaining a sample from a subject,
measuring the level of a biomarker of collagen synthesis (e.g. PRO-C3) in the sample and measuring the level of a biomarker of collagen degradation (e.g. C3M) in the sample,
evaluating the ratio of the level of the biomarker of collagen synthesis (e.g. PRO-C3) to the level of the biomarker of collagen degradation (e.g. C3M) in a subject,
comparing the ratio against a baseline or a reference ratio,
wherein an increase in the ratio relative to the baseline or the reference ratio identifies that the subject is in need of treatment with the neutrophil elastase inhibitor.

### BRIEF DESCRIPTION OF THE FIGURES

FIG. 1 shows the percent change from baseline (% CFB) of plasma desmosine (DES) and isodesmosine (IDES) in patients (N=7) over an escalating dose (0-240 mg) of alvelestat with time (weeks). On the x-axis, 0 (mg) represents the baseline (i.e. pre-treatment at week 0), 60 mg was administered at 2 weeks, 120 mg was administered at 4 weeks, 180 mg was administered at 6 weeks, and 240 mg was administered at 8 weeks. The boxes=mean change from baseline, and the whiskers=the standard deviation.

FIG. 2 shows the percent change from baseline (% CFB) of neutrophil elastase activity in patient blood samples (N=7) over time (wk=week) with increasing doses (0-240 mg) of alvelestat. Assessed ex vivo by measuring neutrophil elastase release in response to zymosan stimulation.

FIG. 3A shows the PRO-C3 level (ng/mL) over time as measured in subjects undergoing alvelestat treatment. ULN=Upper Limit of Normal (i.e. the upper limit of the reference range). Subjects were tested every two weeks.

FIG. 3B shows the PRO-C6 level (ng/ml) over time as measured in subjects undergoing alvelestat treatment. Subjects were tested every two weeks.

FIG. 4 shows the ratio of PRO-C3:C3M in blood samples over time in subjects undergoing alvelestat treatment (n=7). Subjects were tested every two weeks during alvelestat dose escalation.

FIG. 5 shows the % change from baseline (% CFB) of PRO-C3 and DES/IDES in blood samples from each subject (n=7) over the duration of the study. Subjects were tested every two weeks during alvelestat dose escalation.

### DETAILED DESCRIPTION

The description below is made with the understanding that the present disclosure is to be considered as an exemplification of the claimed subject matter, and is not intended to limit the appended claims to the specific embodiments illustrated. The present disclosure provides reference to various embodiments and techniques. However, it should be understood that many variations and modifications may be made while remaining within the spirit and scope of the present disclosure. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. Throughout this specification and in the claims that follow, the following terms are defined with the following meanings, unless explicitly stated otherwise.

Unless the context requires otherwise, throughout the present specification and claims, the word “comprise” and variations thereof, such as, “comprises” and “comprising” are to be construed in an open, inclusive sense, i.e. as “including, but not limited to”.

Where the plural form is used for compounds, salts, and the like, this is taken to mean also a single compound, salt, or the like.

As used herein, the term “or” is generally employed in the sense as including “and/or” unless the context of the usage clearly indicates otherwise.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about” means the recited value±10% of the recited value.

As used herein, “treatment” or “treating” is an approach for obtaining beneficial or desired results. For purposes of the present invention, beneficial or desired results include, but are not limited to, alleviation of a symptom and/or diminishment of the extent of a symptom associated with a disease or condition. “Treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject. The subject may be an individual at risk of developing the disease or disorder, such as an individual who has one or more risk factors known to be associated with development or onset of the disease or disorder. Thus, the term “preventing” in the present invention thus includes administering to a subject who will undergo transplantation, or has recently undergone transplantation without yet developing the associated condition.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease. The effective amount will vary depending on the particular compound, and characteristics of the subject to be treated, such as age, weight, etc. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

The term “solvate” is used herein to describe a molecular complex comprising the compound of the invention and a one or more pharmaceutically acceptable solvent molecules, for example, ethanol or water. The term “hydrate” is employed when the solvent is water and for the avoidance of any doubt, the term “hydrate” is encompassed by the term “solvate”.

The term “pharmaceutically acceptable salt” means a physiologically or toxicologically tolerable salt and includes, when appropriate, pharmaceutically acceptable base addition salts and pharmaceutically acceptable acid addition salts. For example, where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like. Hemisalts of acids and bases can also be formed, for example, hemisulfate and hemicalcium salts. For a review of suitable salts, see “Handbook of Pharmaceutical Salts: Properties, Selection and Use” by Stahl and Wermuth (Wiley-VCH, 2011).

“Pharmaceutically acceptable” or “physiologically acceptable” refer to compounds, salts, compositions, dosage forms etc. which are suitable for pharmaceutical use.

The term “subject” preferably refers to a human, normally who has received a transplant or is about to receive a transplant.

All documents referenced herein are each incorporated by reference in their entirety for all purposes.

### Alvelestat

The preferred neutrophil elastase inhibitor used in the invention is alvelestat.

Alvelestat is a potent, orally bioavailable neutrophil elastase inhibitor described in WO 2005/026123 A1 (Example 94, page 85) and [6], which are incorporated herein by reference in their entirety. Alvelestat has the chemical name N-{[5-(methanesulfonyl) pyridin-2-yl]methyl}-6-methyl-5-(1-methyl-1H-pyrazol-5-yl)-2-oxo-1-[3-(trifluoromethyl) phenyl]-1,2-dihydropyridine-3-carboxamide, and the following chemical structure:

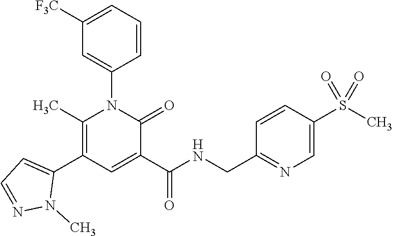 [SMILES: Cc1c(-c2ccnn2C)cc(C(=O)NCc2ccc(S(C)(=O)=O)cn2)c(=O)n1-c1cccc(C(F)(F)F)c1]

Alvelestat has also been referred to as AZD9668 and MPH996.

Alvelestat may be used in the invention in any pharmaceutically acceptable form, for example any free base form, salt form, and/or solvate form. Alvelestat or a pharmaceutically acceptable salt and/or solvate thereof may be present in any pharmaceutically acceptable physical form, suitably a solid form.

Certain salts of alvelestat are described in WO 2010/094964 A1, which is incorporated herein by reference in its entirety. Described salts of alvelestat include the tosylate, p-xylene-2-sulfonate, chloride, mesylate, esylate, 1,5-naphthalenedisulfonate and sulfate.

Preferably, alvelestat free base or alvelestat tosylate is used in the methods of the invention, more preferably alvelestat tosylate.

Alvelestat may also be used in any of the methods of the invention in a pharmaceutically acceptable prodrug form.

### Neutrophil Elastase Inhibitors

Neutrophil elastase (NE) is an enzyme that attacks and progressively damages lung tissue. Compounds that inhibit NE are reviewed in [7] and are known from various publications including WO2017207430, WO2017102674, WO2016050835, WO2016050835, WO2016016368, WO2016016366, WO2016016365, WO2016016364, WO2016016363, WO2015124563, WO2016020070, WO2015091281, WO2014135414, WO2014122160, WO2015096873, WO2015096872, WO2014029832, WO2014029831, WO2014029830, WO2014009425, WO2013084199, WO2013037809, WO2011103774, WO2011110858, WO2011110859, WO2011110852, WO2011039528, WO2010034996, WO2009061271, WO2009058076, WO2009060206, WO2007137080, WO2007137080, WO2007140117, WO2008036379, WO2008036379, WO9962538, WO9962538, WO9962514, WO9739028, WO9616080, WO9533763, WO9533762, WO9527055, WO9311760, WO9220357, WO9215605, WO9215605, WO03058237, WO03031574, WO03031574, WO2008030158, WO2007129963, WO2007129962, WO2006098684, WO2005026124, WO2005026123, WO2005021509, WO2005021512, WO2004043924, WO2009060158, WO2009037413, WO2009013444, WO2007129060, WO2007107706, WO2007107706, WO2006136857, WO2006082412, WO2006082412, WO9623812, WO9521855, WO9401455, WO9324519, WO9321214, WO9321210, WO9321213, WO9321209, WO9321212, WO2006070012, WO2005082863, WO2005082863, WO2005082864, WO9912933, WO9912933, WO9912931, WO9736903, WO2004020412, WO2008104752, WO2008097676, WO200809767, WO2008085608, each of which is incorporated by reference. Each of the neutrophil inhibitors described in these publications may be used in the methods of the invention, and is referred to as if it were individually disclosed herein for use in the methods of the invention.

In addition to the preferred neutrophil elastase inhibitor alvelestat, other exemplary neutrophil elastase inhibitors that may be used in the present invention include sivelestat, ONO-5046-Na, depelestat, Prolastin, KRP-109, DX-890, pre-elafin, MNEI, BAY 85-8501, POL6014, α1-AT, sirtinol, ONO-6818 (2-(5-amino-6-oxo-2-phenyl-1,6-dihydro-pyrimidin-1-yl)-N-[(1R,2R)-1-(5-tert-butyl-1,3,4-oxadiazol-2-yl)-1-hydroxy-3-methylbutan-2-yl]acetamide), elastatinal, SSR 69071 (2-[6-methoxy-4-(1-methylethyl)-1,1-dioxo-3-oxo-1,2-benzisothiazol-2(3H)-yl] methoxy]-9-[2-(1-piperidinyl)ethoxy]-4H-pyrido[1,2-a]pyrimidin-4-one), and M0398 (N-(methoxysuccinyl)-L-alanyl-L-alanyl-L-prolyl-L-valine chrolomethylketone); and their pharmaceutically acceptable salts and/or solvates. In addition to the preferred neutrophil elastase inhibitor alvelestat, other exemplary neutrophil elastase inhibitors that may be used in the present invention include sivelestat, ONO-5046-Na, depelestat, Prolastin, KRP-109, DX-890, pre-elafin, MNEI, BAY-85-8501 (also referred to as PHP-303), BAY-678, DMP-777, GW-311616, POL6014, α1-AT, sirtinol, ONO-6818 (2-(5-amino-6-oxo-2-phenyl-1,6-dihydro-pyrimidin-1-yl)-N-[(1R, 2R)-1-(5-tert-butyl-1,3,4-oxadiazol-2-yl)-1-hydroxy-3-methylbutan-2-yl]acetamide), elastatinal, SSR 69071 (2-[[6-methoxy-4-(1-methylethyl)-1,1-dioxo-3-oxo-1,2-benzisothiazol-2(3H)-yl] methoxy]-9-[2-(1-piperidinyl)ethoxy]-4H-pyrido[1,2-a]pyrimidin-4-one), and M0398 (N-(methoxysuccinyl)-L-alanyl-L-alanyl-L-prolyl-L-valine chrolomethylketone); and their pharmaceutically acceptable salts and/or solvates. In certain embodiments, the neutrophil elastase inhibitor used in the methods of the invention is a specific and reversible inhibitor of NE, such as ONO-6818 (also known as freselestat). In certain embodiments, the neutrophil elastase inhibitor used in the methods of the invention is an irreversible inhibitor of NE, such as DMP-777 (CAS number: 157341-41-8). In certain embodiments, the neutrophil elastase inhibitor used in the methods of the invention is an inhibitor of NE and PR3, such as: sivelestat, BAY-678 (CAS number: 675103-36-3), BAY-85-8501 (CAS number: 1161921-82-9), DMP-777 (CAS number: 157341-41-8), or GW-311616 (CAS number: 198062-54-3).

The term neutrophil elastase inhibitor includes all pharmaceutically acceptable forms of the compounds, for example all pharmaceutically acceptable salt, solvate, isomer, and prodrug forms.

In certain embodiments, the neutrophil elastase inhibitor is a small molecule compound, i.e. has a molecular weight of less than about 900 Daltons.

Preferably the neutrophil elastase inhibitors are inhibitors of human neutrophil elastase.

Although many embodiments of this invention relate to alvelestat, it should be understood that for each and every embodiment described herein referring to “alvelestat”, the invention also provides a corresponding embodiment involving the use of “a neutrophil elastase inhibitor”.

### Treatments

The invention generally provides methods for treating or preventing fibrosis, fibrosis associated with a tissue, or fibrosis associated with a disease (e.g. graft rejection, lung transplant associated bronchiolitis obliterans syndrome (LT-BOS), graft versus host disease (GVHD), or GVHD associated bronchiolitis obliterans syndrome (BOS)) etc., in a subject in need thereof comprising administering to the subject an effective amount of a neutrophil elastase inhibitor, in particular alvelestat or a pharmaceutically acceptable salt and/or solvate thereof.

Accordingly, the present invention provides a method for treating or preventing fibrosis, comprising administering an effective amount of alvelestat or a pharmaceutically acceptable salt and/or solvate thereof to a subject in need thereof.

The present invention further provides a method for reducing fibrosis in a subject, comprising administering an effective amount of alvelestat or a pharmaceutically acceptable salt and/or solvate thereof to a subject in need thereof.

In certain embodiments, the fibrosis can be arthrofibrosis. Arthrofibrosis is a form of scar tissue formation (i.e. fibrosis) that can occur in one or more of knee, hip, ankle, foot joints, shoulder (frozen shoulder, adhesive capsulitis), elbow (stiff elbow), wrist, hand joints, and spinal vertebrae.

In certain embodiments, the fibrosis can be liver fibrosis. Liver fibrosis can be cirrhosis which refers to the scar tissue and nodules that replace liver tissue and disrupt liver function. Liver fibrosis is usually caused by alcoholism, fatty liver disease, hepatitis B or hepatitis C. Liver fibrosis can be caused by fatty liver disease which is also known as non-alcoholic fatty liver disease (NAFLD). In certain embodiments, the fibrosis can be liver fibrosis associated with non-alcoholic fatty liver disease (NAFLD). In certain embodiments, the fibrosis can be associated with non-alcoholic fatty liver disease (NAFLD). In certain embodiments, NAFLD can comprise one or more of non-alcoholic steatosis (e.g. simple fatty liver or steatosis), non-alcoholic steatohepatitis (NASH), fibrosis and cirrhosis. In certain embodiments, the fibrosis can be liver fibrosis associated with non-alcoholic steatohepatitis (NASH). In certain embodiments, the fibrosis can be associated with non-alcoholic steatohepatitis (NASH).

In certain embodiments, the fibrosis can be hepatic fibrosis. Hepatic fibrosis can be result of the wound-healing response of the liver to repeated injury. For example, after a liver injury (e.g. caused by viral hepatitis), parenchymal cells can regenerate and replace the necrotic or apoptotic cells. This process can be associated with an inflammatory response and the deposition of ECM. If the hepatic injury persists, then eventually the liver regeneration fails, and hepatocytes can be substituted with abundant ECM, leading to fibrosis.

In certain embodiments, the fibrosis can be heart fibrosis. Heart fibrosis can mean areas of the heart that have become damaged due to myocardial infarction which undergo fibrosis. Heart fibrosis is also known as myocardial fibrosis. Myocardial fibrosis has two forms. The first is interstitial fibrosis, which can be a result of congestive heart failure, hypertension, and aging. The second is replacement fibrosis, which can be a result of older myocardial infarction. In certain embodiments, the myocardial fibrosis is interstitial fibrosis or replacement fibrosis.

In certain embodiments, the fibrosis can be mediastinal fibrosis. Mediastinal fibrosis is a form of fibrosis is characterized by calcified fibrosis of the lymph nodes, which can block respiratory channels and blood vessels.

In certain embodiments, the fibrosis can be retroperitoneal cavity fibrosis. Retroperitoneal cavity fibrosis is fibrosis of the soft tissue in the retroperitoneum, which contains the aorta, kidneys and numerous other structures.

In certain embodiments, the fibrosis can be bone marrow fibrosis. Bone marrow fibrosis, or myelofibrosis, is scarring in the bone marrow that prevents the normal production of blood cells in the bone marrow.

In certain embodiments, the fibrosis can be bridging fibrosis. Bridging fibrosis is a type of fibrosis seen in several types of liver injury, and describes fibrosis from the portal vein to the portal triad.

In certain embodiments, the fibrosis can be nephrogenic systemic fibrosis. Nephrogenic systemic fibrosis can involve fibrosis of skin, joints, eyes, and/or internal organs.

In certain embodiments, the fibrosis can be skin fibrosis. Skin fibrosis is scar tissue that forms on the skin in response to injury, and can also be referred to as a keloid.

In certain embodiments, the fibrosis can be scleroderma or systemic sclerosis. Scleroderma is an autoimmune disease of the connective tissue that primarily affects the skin but can also involve other organs such as the kidneys, heart and/or lungs.

The present invention further provides a method for treating or preventing fibrosis in a tissue, comprising administering an effective amount of alvelestat or a pharmaceutically acceptable salt and/or solvate thereof to a subject in need thereof. In certain embodiments, the tissue is lung tissue. In certain embodiments, the tissue is liver tissue. In certain embodiments, the tissue is brain tissue. In certain embodiments, the tissue is heart tissue. In certain embodiments, the tissue is gastrointestinal tissue. In certain embodiments, the tissue is skin tissue.

In certain embodiments of the method for treating or preventing fibrosis in a tissue, the tissue is also associated with GVHD. Thus, the present invention further provides a method for treating or preventing fibrosis in a tissue associated with GVHD, comprising administering an effective amount of alvelestat or a pharmaceutically acceptable salt and/or solvate thereof to a subject in need thereof. In certain embodiments, the tissue is selected from the group comprising liver, brain, heart, gastrointestinal tissue and skin tissue. In certain embodiments, the tissue is lung tissue. In certain embodiments, the tissue is liver tissue. In certain embodiments, the tissue is brain tissue. In certain embodiments, the tissue is heart tissue. In certain embodiments, the tissue is gastrointestinal tissue. In certain embodiments, the tissue is skin tissue.

The present invention further provides a method for treating or preventing fibrosis associated with a disease, comprising administering an effective amount of alvelestat or a pharmaceutically acceptable salt and/or solvate thereof to a subject in need thereof.

In certain embodiments, the disease can be selected from graft rejection, graft versus host disease (GVHD), chronic lung allograft dysfunction (CLAD), Lung Transplant associated Bronchiolitis Obliterans Syndrome (LT-BOS), bronchiolitis obliterans syndrome (BOS), GVHD associated BOS (GVHD BOS), Lung Transplant associated Restrictive Allograft Syndrome (LT-RAS), GVHD associated restrictive chronic lung function decline (GVHD R-CLFD), liver disease, heart disease, cirrhosis, fibrothorax, radiation-induced lung injury, glial scar, arterial stiffness, kidney disease, Crohn's disease, Dupuytren's contracture, keloid disorder, Peyronie's disease and adhesive capsulitis, rheumatoid arthritis, ulcerative colitis, systemic lupus erythematosus, and hypertension. In particular, the disease can be selected from LT-BOS, GVHD BOS, and BOS. In certain embodiments, the disease can be selected from non-alcoholic fatty liver disease (NAFLD), non-alcoholic steatosis, non-alcoholic steatohepatitis (NASH), and cirrhosis.

Fibrothorax can be characterised by severe scarring (fibrosis) and fusion of the layers of the pleural space surrounding the lungs resulting in decreased movement of the lung and ribcage. A glial scar can be the formation (gliosis) defined as the reactive cellular process involving astrogliosis that occurs after injury to the central nervous system. Arterial stiffness can occur as a consequence of biological aging and/or arteriosclerosis. Dupuytren's contracture (also called Dupuytren's disease, Morbus Dupuytren, Viking disease, and Celtic hand) is a condition in which one or more fingers become permanently bent in a flexed position. Restrictive chronic lung function decline (R-CLFD) can be defined by the restrictive decline in pulmonary function and can involve fibrotic changes in the alveolar units.

The present invention further provides a method for treating or preventing fibrosis associated with graft rejection, comprising administering an effective amount of alvelestat or a pharmaceutically acceptable salt and/or solvate thereof to a subject in need thereof.

The present invention further provides a method for treating or preventing graft rejection, comprising administering an effective amount of alvelestat or a pharmaceutically acceptable salt and/or solvate thereof to a subject in need thereof, wherein the treatment comprises reducing fibrosis in the subject.

Graft rejection may also be referred to as organ transplant rejection.

A subject with acute graft rejection (such as acute lung rejection) has an increased risk of developing chronic graft rejection (such as CLAD and in particular LT-BOS). Thus, the present invention further provides a method for preventing development of fibrosis in a subject with acute graft rejection, comprising administering an effective amount of alvelestat or a pharmaceutically acceptable salt and/or solvate thereof to the subject in need thereof.

The methods described herein are useful for treating or preventing fibrosis associated with chronic graft rejection. In certain embodiments, the method is for treating fibrosis associated with chronic graft rejection. In other embodiments, the method is for preventing fibrosis associated with chronic graft rejection.

The graft may comprise any solid organ, in particular those solid organs that are frequently transplanted. So, the graft may comprise one or more organs selected from the group consisting of skin, kidney, heart, liver, gastrointestinal tract, lung and pancreas.

Chronic rejection of cardiac (i.e. heart) allografts is manifested by cardiac allograft vasculopathy (CAV). This is typically characterized by occlusion of coronary vessels. The 5-year incidence of CAV is 30-40%. Accordingly, the present invention provides a method for treating or preventing CAV in a subject in need thereof comprising administering to the subject an effective amount of alvelestat or a pharmaceutically acceptable salt and/or solvate thereof.

Chronic rejection of kidney allografts is manifested by cardiac allograft nephropathy (CAN). CAN is the leading cause of renal function deterioration and accounts for nearly 40% of the graft loss at 10 years. Accordingly, the present invention provides a method for treating or preventing CAN in a subject in need thereof comprising administering to the subject an effective amount of alvelestat or a pharmaceutically acceptable salt and/or solvate thereof.

In preferred embodiments, the graft comprises a lung. The graft may be a single or a double lung transplant. The graft may be a heart-lung transplant. Thus, the present invention provides a method for treating or preventing fibrosis associated with lung transplant rejection in a subject in need thereof comprising administering to the subject an effective amount of alvelestat or a pharmaceutically acceptable salt and/or solvate thereof. It may be chronic lung transplant rejection.

Chronic rejection of lung allografts is manifested by chronic lung allograft dysfunction (CLAD).

Accordingly, the present invention provides a method for treating or preventing fibrosis associated with CLAD, comprising administering an effective amount of alvelestat or a pharmaceutically acceptable salt and/or solvate thereof, to a subject in need thereof.

The present invention further provides a method for treating or preventing CLAD, comprising administering an effective amount of alvelestat or a pharmaceutically acceptable salt and/or solvate thereof, to a subject in need thereof, wherein the treatment comprises reducing fibrosis in the subject.

A phenotype of CLAD is lung transplant associated bronchiolitis obliterans syndrome (LT-BOS). Bronchiolitis obliterans may also be referred to as obliterative bronchiolitis. Fibrosis can be associated with bronchiolitis obliterans. Typical characteristics include an obstructive pulmonary function defect and air trapping/mosaic attenuation on expiratory CT.

The present invention provides a method for treating or preventing fibrosis associated with LT-BOS, comprising administering an effective amount of alvelestat or a pharmaceutically acceptable salt and/or solvate thereof, to a subject in need thereof.

The present invention further provides a method for treating or preventing LT-BOS, comprising administering an effective amount of alvelestat or a pharmaceutically acceptable salt and/or solvate thereof to a subject in need thereof, wherein the treatment comprises reducing fibrosis in the subject.

Another phenotype of CLAD is lung transplant associated Restrictive Allograft Syndrome (RAS). Typical characteristics include peripheral lung fibrosis.

The present invention provides a method for treating or preventing fibrosis associated with LT-RAS, comprising administering an effective amount of alvelestat or a pharmaceutically acceptable salt and/or solvate thereof, to a subject in need thereof.

The present invention further provides a method for treating or preventing LT-RAS, comprising administering an effective amount of alvelestat or a pharmaceutically acceptable salt and/or solvate thereof to a subject in need thereof, wherein the treatment comprises reducing fibrosis in the subject.

Methods of preventing fibrosis associated with LT-BOS and/or LT-RAS according to the invention are particularly useful for subjects at risk of LT-BOS and/or LT-RAS. Such subjects may possess one or more risk factors selected from the group consisting of primary graft dysfunction, gastro-oesophageal reflux, infection, airway ischemia, acute rejection, lymphocytic bronchiolitis, infection and colonization with micro-organisms (e.g., Pseudomonas aeruginosa and Aspergillus fumigatus), donor and recipient genetics, particulate matter and presence of HLA antibodies, or antibodies to self-antigens (such as K-a1 tubulin and collagen V).

The present invention provides a method for treating or preventing fibrosis associated with BOS, comprising administering an effective amount of alvelestat or a pharmaceutically acceptable salt and/or solvate thereof, to a subject in need thereof.

The present invention further provides a method for treating or preventing BOS, comprising administering an effective amount of alvelestat or a pharmaceutically acceptable salt and/or solvate thereof to a subject in need thereof, wherein the treatment comprises reducing fibrosis in the subject.

The present invention provides a method for treating or preventing fibrosis associated with GVHD, comprising administering an effective amount of alvelestat or a pharmaceutically acceptable salt and/or solvate thereof, to a subject in need thereof.

The present invention provides a method for treating or preventing graft versus host disease (GVHD) comprising administering an effective amount of alvelestat or a pharmaceutically acceptable salt and/or solvate thereof in a subject in need thereof, wherein the treatment comprises reducing fibrosis in the subject.

Fibrosis with GVHD can manifest following tissue transplantation. In some embodiments, the transplant is selected from the group consisting of skin, hematopoietic stem cells, blood and bone marrow. In preferred embodiments, the transplant is hematopoietic stem cells. In certain embodiments, the transplant is hematopoietic stem cells, for example an allogeneic hematopoietic cell transplant.

The GVHD may be acute graft versus host disease (aGVHD). The disease may be chronic graft versus host disease (cGVHD). Acute GVHD is typically characterized by damage to the skin, liver and the gastrointestinal tract, whereas chronic GVHD typically has more diverse manifestations and can resemble autoimmune syndromes with, for example, eosinophilic fasciitis, scleroderma-like skin disease and salivary and lacrimal gland involvement.

An additional embodiment provides a method for inhibiting the onset of symptoms, such as fibrosis, of GVHD, including aGVHD and cGVHD, the method comprising administering a pharmaceutically effective amount of alvelestat or a pharmaceutically acceptable salt and/or solvate thereof to a recipient of a transplantation of allogenic hematopoietic stem cells.

In the above methods relating to GVHD, the GVHD may be characterised by damage to one or more selected from the group consisting of the eyes, joints, fascia, genital organ, lung, liver, skin, or gastrointestinal tract (e.g. mouth, oesophagus).

In particular, in the above methods relating to GVHD, the GVHD may be characterised by damage to one or more selected from the group consisting of the lung, liver, skin, or gastrointestinal tract. In particular, in the above methods relating to GVHD, the GVHD may be characterised by damage to one or more selected from the group consisting of the liver, skin or gastrointestinal tract.

Chronic GVHD may be classified according to various criteria. The 2005 and 2014 National Institutes of Health Consensus Development Projects on Criteria for Clinical Trials in Chronic GVHD standardized the terminology around chronic GVHD classification systems [8].

One classification system is the NIH severity score, which is divided into mild, moderate or severe based on the number and severity of involved organs. Accordingly, in methods of the invention relating to treating cGVHD, the subject may have cGVHD which is mild, moderate or severe according to the NIH severity score. In particular, the cGVHD may be moderate or severe, typically severe. Furthermore, methods for improving the cGVHD severity score in a subject with cGVHD are provided herein, comprising administering alvelestat or a pharmaceutically acceptable salt and/or solvate thereof.

Another classification system based on patient-reported outcomes is the Lee cGVHD Symptom Scale [9]. Accordingly, methods for improving the Lee cGVHD Symptom Scale in a patient with cGVHD are provided herein, comprising administering alvelestat or a pharmaceutically acceptable salt and/or solvate thereof. In particular, methods for improving the Lee cGVHD Symptom Scale lung score in a subject with cGVHD affecting a lung are provided herein, comprising administering alvelestat or a pharmaceutically acceptable salt and/or solvate thereof.

Bronchiolitis Obliterans Syndrome (BOS) is a rare but devastating complication of graft-versus-host disease (GVHD), and this complication can be referred to as GVHD BOS (or GVHD-associated BOS).

The invention also provides a method for treating or preventing fibrosis associated with GVHD BOS, comprising administering an effective amount of alvelestat or a pharmaceutically acceptable salt and/or solvate thereof to a subject in need thereof.

The invention also provides a method for treating or preventing GVHD BOS, comprising administering an effective amount of alvelestat or a pharmaceutically acceptable salt and/or solvate thereof to a subject in need thereof, wherein the treatment comprises reducing fibrosis in the subject.

In preferred embodiments, the subject has received a hematopoietic stem cell transplantation, such as an allogeneic hematopoietic cell transplant.

In preferred embodiments, the subject is a human subject.

Thus, invention provides a method for treating or preventing fibrosis associated with GVHD BOS, comprising administering an effective amount of alvelestat or a pharmaceutically acceptable salt and/or solvate thereof to a human subject in need thereof, wherein the subject has received a hematopoietic cell transplant.

In the above methods relating to GVHD BOS, the GVHD may be characterised by damage to one or more selected from the group consisting of the eyes, joints, fascia, genital organ, lung, liver, skin, or gastrointestinal tract (e.g. mouth, oesophagus). In particular, in the above methods relating to GVHD BOS, the GVHD may be characterised by damage to one or more selected from the group consisting of the liver, skin, or gastrointestinal tract.

In certain embodiments, the subject has neutrophilia, for example, airway neutrophilia.

Also provided is alvelestat or a pharmaceutically acceptable salt thereof for use in treating or preventing fibrosis associated with GVHD BOS. In particular, also provided is alvelestat or a pharmaceutically acceptable salt thereof for use in treating or preventing fibrosis associated with GVHD BOS in a human subject, wherein the human subject has received a hematopoietic cell transplant.

Also provided is the use of alvelestat or a pharmaceutically acceptable salt thereof for the manufacture of a medicament for treating or preventing fibrosis associated with GVHD BOS. In particular, also provided is the use of alvelestat or a pharmaceutically acceptable salt thereof for the manufacture of a medicament for treating or preventing fibrosis associated with GVHD BOS in a human subject, wherein the human subject has received a hematopoietic cell transplant.

Restrictive chronic lung function decline (R-CLFD) is also a devastating complication of graft-versus-host disease (GVHD), and this complication can be referred to as GVHD R-CLFD (or GVHD-associated R-CLFD).

The invention also provides a method for treating or preventing fibrosis associated with GVHD R-CLFD, comprising administering an effective amount of alvelestat or a pharmaceutically acceptable salt and/or solvate thereof to a subject in need thereof.

The invention also provides a method for treating or preventing GVHD R-CLFD, comprising administering an effective amount of alvelestat or a pharmaceutically acceptable salt and/or solvate thereof to a subject in need thereof, wherein the treatment comprises reducing fibrosis in the subject.

In preferred embodiments, the subject has received a hematopoietic stem cell transplantation, such as an allogeneic hematopoietic cell transplant.

In preferred embodiments, the subject is a human subject.

In some embodiments, the treatment, prevention or reduction of fibrosis comprises reducing the level of one or more biomarkers. In some embodiments, the subject has elevated levels of one or more biomarkers, for example the subject has elevated levels of one or more biomarkers prior to administering an NE inhibitor. In some embodiments, the subject has elevated NE activity, for example the subject has elevated NE activity prior to administering an NE inhibitor. In some embodiments, the treatment, prevention, or reduction of fibrosis comprises reducing the level of one or more biomarkers over 8 weeks.

During collagen turnover, distinct peptides are generated as by-products of both collagen synthesis and degradation. By-products of collagen synthesis include the propeptide of pro-collagen which is cleaved before collagen is incorporated into the extracellular matrix.[10][11] This results in the release of unique fragments (e.g. neoepitopes) for each individual form of collagen which can be detected in the blood. By-products of collagen degradation arise from specific metalloproteases (MMPs) which cleave collagen fibres revealing distinct neo-epitopes. Both synthesis peptides and degradation neo-epitopes are released into the circulation and can be detected in the blood. Ratios of collagen synthesis to collagen degradation by-products (e.g. biomarkers) are useful indicators of collagen remodelling and thus fibrosis. For example, a decrease in the ratio of the level of collagen synthesis by-products to the level of collagen degradation by-products can be associated with a decrease in fibrosis.

In some embodiments, the biomarker is a collagen biomarker. In some embodiments, the biomarker is a collagen synthesis biomarker. In some embodiments, the collagen synthesis biomarker is a type III collagen biomarker and/or a type VI collagen biomarker. In some embodiments, the collagen synthesis biomarker is a type III collagen biomarker and a type VI collagen biomarker. In some embodiments, the type III collagen biomarker is the N-terminal propeptide of type III collagen (PRO-C3). In some embodiments, the type VI collagen biomarker is the N-terminal propeptide of type VI collagen (PRO-C6). In some embodiments, the biomarker is PRO-C3. In some embodiments, the biomarker is PRO-C6.

In healthy volunteers, the plasma PRO-C3 level (i.e. a reference level) was approximately 10+/−2 ng/ml as measured by competitive ELISA.[1] In some embodiments, the reference level of PRO-C3 about 8-12, about 9-11 or about 10 ng/ml. In some embodiments, the reference level is measured by competitive ELISA.

In healthy volunteers, the plasma PRO-C6 level (i.e. a reference level) was approximately 8+/−1 ng/ml as measured by competitive ELISA.[1] In some embodiments, the reference level of PRO-C3 about 7-9, or about 8 ng/ml. In some embodiments, the reference level is measured by competitive ELISA.

In some embodiments, the treatment, prevention or reduction of fibrosis comprises reducing the level of PRO-C3 and/or PRO-C6.

In some embodiments, the biomarker is a collagen degradation biomarker. In some embodiments, the collagen degradation biomarker is a type III collagen biomarker (C3M) and/or a type VI collagen biomarker (C6M). In some embodiments, the collagen biomarker is a type III collagen biomarker (C3M) and a type VI collagen biomarker (C6M). In some embodiments, the biomarker is C3M. In some embodiments, the biomarker is C6M.

In some embodiments, the biomarker is an elastin biomarker. In some embodiments the elastin biomarker is selected from desmosine (DES) and isodesmosine (IDES). In some embodiments the elastin biomarker is desmosine (DES). In some embodiments the elastin biomarker is isodesmosine (IDES).

In healthy volunteers, the plasma DES/IDES level (i.e. a reference level) was approximately 0.2 ng/ml using LC-MS/MS. The plasma DES/IDES level refers to the total concentration of DES and IDES. Thus, in some embodiments, the level of desmosine (DES) and isodesmosine (IDES) is the total level of desmosine (DES) and isodesmosine (IDES). In some embodiments, the reference level of desmosine (DES) and isodesmosine (IDES) is 0.2 ng/ml. In some embodiments, the reference level is measured by LC-MS/MS.

Thus, in some embodiments, the treatment, prevention or reduction of fibrosis comprises reducing the level of desmosine (DES) and isodesmosine (IDES).

Thus, in some embodiments, the treatment, prevention or reduction of fibrosis comprises reducing the level of desmosine (DES), isodesmosine (IDES), PRO-C3 and PRO-C6.

Thus, in some embodiments, the treatment, prevention or reduction of fibrosis comprises reducing the level of desmosine (DES), isodesmosine (IDES), and PRO-C3.

Thus, in some embodiments, the treatment, prevention or reduction of fibrosis comprises a decrease in the ratio of PRO-C3:C3M. In some embodiments, the treatment, prevention or reduction of fibrosis comprises a decrease in the ratio of PRO-C6:C6M.

The reduction in level of any of the biomarkers described herein (e.g. one or more of PRO-C3, PRO-C6, DES and IDES, C3M, and C6M) can be determined by a method (e.g. an ex vivo method). For example, the level of a biomarker can be determined by taking a blood sample from a subject and evaluating the sample in an ex vivo method. The reduction in a level of a biomarker can be determined by establishing a baseline or reference level of the biomarker, prior to administering an NE inhibitor described herein, and then determining the level of the biomarker after administering an NE inhibitor described herein.

In the described methods, the NE inhibitor, in particular alvelestat or a pharmaceutically acceptable salt and/or solvate thereof, may be administered to the subject prior to transplantation. For example, alvelestat administration may start 14 days, 7 days, 3 days, 2 days, or 1 day prior to transplantation.

In the described methods, the NE inhibitor, in particular alvelestat or a pharmaceutically acceptable salt and/or solvate thereof, may be administered to the subject after transplantation. For example, the alvelestat administration may start on the day of transplantation or 1 day, 2 days, 3 days, 7 days, or 14 days after transplantation.

Methods according to the invention relating to BOS, in particular LT-BOS or GVHD BOS, may also comprise improving one or more pulmonary function parameters in a subject. Methods according to the invention relating to restrictive lung disease, in particular LT-RAS, R-CLFD or GVHD R-CLFD, may also comprise improving one or more pulmonary function parameters in a subject.

In particular, methods according to the invention may improve the FEV1 of a subject. Forced expiratory volume (FEV) is the expiratory volume of air from a maximally forced effort measured over a set period of time, e.g. 1 second (FEV1).

In particular, methods according to the invention may improve the FEV1% predicted of a subject. FEV1% predicted is the ratio of FEV1 in a subject to the predicted FEV1 of a normal person of similarly matched race or ethnicity, gender, age, height and weight, expressed as a percentage.

Accordingly, also provided is a method for increasing FEV1% predicted in a subject with fibrosis and LT-BOS, by administering an effective amount of a NE inhibitor, in particular alvelestat or a pharmaceutically acceptable salt and/or solvate thereof.

Accordingly, also provided is a method for increasing FEV1% predicted in a subject with fibrosis and GVHD BOS, by administering an effective amount of a NE inhibitor, in particular alvelestat or a pharmaceutically acceptable salt and/or solvate thereof.

In particular embodiments, treatment with an effective amount of alvelestat or a pharmaceutically acceptable salt and/or solvate thereof increases FEV1% predicted by at least about 1%, 1.5%, 2.0%, 2.5%, 3.0%, 4.0%, 5.0%, 6.0%, 7.0%, 8.0%, 9.0%, 10%, 15%, 20%, 30%, 40% or 50% compared to a baseline FVC % predicted measurement. In further embodiments, treatment with an effective amount of alvelestat or a pharmaceutically acceptable salt and/or solvate thereof prevents FEV1% from worsening.

Methods according to the invention relating to LT-BOS or GVHD BOS may also comprise improving the BOS grade of a subject. The BOS classification scheme, adopted in 1993, provided a staging system based on the severity of lung function decline after transplant and has been used for clinical decision-making and research purposes. This staging system was most recently modified in 2002 [2]. The BOS classification scheme from 2002 is used according to the invention:

Grading (staging) of bronchiolitis obliterans syndrome (BOS)^{#}

| BOS grade | Spirometry % of baseline^{¶} |
|---|---|
| 0 | FEV₁ > 90% and FEF₂₅₋₇₅% > 75% |
| 0-p⁺ | FEV₁ 81-90% and/or FEF_{25-75%} ≤ 75% |
| 1 | FEV₁ 66-80% |
| 2 | FEV₁ 51-65% |
| 3 | FEV₁ ≤ 50% |

FEV1: forced expiratory volume in 1 s; FEF_{25-75%}: forced expiratory flow at 25-75% of forced vital capacity.
^{#}Other causes of lung function decline must be excluded (e.g. acute rejection, infection, native lung problems for single lung recipients, excessive recipient weight gain, anastomotic dysfunction, respiratory muscle dysfunction, effusion, or technical problems such as erroneous measurements due to device dysfunction);
^{¶}baseline is defined as the average of the two best FEV₁ (or FEF_{25-75%}) values (≥ 3 weeks apart) following functional recovery and stabilisation post-lung transplantation;
⁺in Grade (stage) 0-p the “p” denotes “probable” early BOS and is used to indicate 10-20% decline in baseline FEV1 that is likely due to an early stage of BOS that does not meet criteria for BOS Grade 1.

Thus in embodiments relating to treatment of fibrosis associated with LT-BOS or GVHD BOS, treatment with an effective amount of alvelestat or a pharmaceutically acceptable salt and/or solvate thereof improves the BOS grading by at least 1 grade. In further embodiments relating to treatment of fibrosis associated with LT-BOS or GVHD BOS, treatment with an effective amount of alvelestat or a pharmaceutically acceptable salt and/or solvate thereof prevents the BOS grading from worsening.

Diagnosis of BOS can be carried out by the skilled clinician. Imaging tests, such as high resolution chest CT scan, and pulmonary function tests can help detect BOS. Chest x-rays may also be used. A surgical lung biopsy can also be carried out to diagnose the BOS. Lung biopsies may show small airway involvement with fibrinous obliteration of the lumen. Bronchoalveolar lavage (BAL) may show neutrophilic and/or lymphocytic inflammation.

Also provided in this invention are methods for treating or preventing fibrosis associated with BOS and connective tissue disease, systemic lupus erythematosus, rheumatoid arthritis, infection, toxic fume exposure, or Stevens-Johnson syndrome, comprising administering an effective amount of a neutrophil elastase inhibitor, in particular alvelestat or a pharmaceutically acceptable salt and/or solvate thereof, to a subject in need thereof.

The patient to be treated in the methods of the present invention may in some embodiments have a baseline FEV1 of 30% of predicted FEV1 or higher, e.g. a baseline FEV1 of 35% or higher, or 40% or higher. The patient may have a baseline FEV1 of 20-90% of predicted FEV1, for example of 30-80%, 35-75%, or 40-50%.

Without wishing to be bound by theory, it is considered that alvelestat is beneficial in the methods of the invention due to its ability to inhibit neutrophil elastase. Accordingly, the invention also provides methods of inhibiting neutrophil elastase in a subject suffering from, or at risk of, any of the conditions described herein, including fibrosis associated with graft rejection, fibrosis associated with GVHD, fibrosis associated with LT-BOS, fibrosis associated with BOS, or fibrosis associated with GVHD BOS, comprising administering an effective amount of a neutrophil elastase inhibitor, in particular alvelestat or a pharmaceutically acceptable salt and/or solvate thereof to the subject.

Thus, the invention provides a method of inhibiting neutrophil elastase in a subject suffering from, or at risk of, fibrosis associated with LT-BOS, BOS, or

GVHD BOS, comprising administering an effective amount of a neutrophil elastase inhibitor, in particular alvelestat or a pharmaceutically acceptable salt and/or solvate thereof to the subject.

In particular, the invention also provides a method of inhibiting neutrophil elastase in a subject suffering from, or at risk of, fibrosis associated with GVHD BOS, comprising administering an effective amount of a neutrophil elastase inhibitor, in particular alvelestat or a pharmaceutically acceptable salt and/or solvate thereof to the subject, wherein the subject has a received a hematopoietic cell transplantation.

Also provided are each of the above methods for treating or preventing any of the conditions described herein by inhibiting neutrophil elastase, comprising administering an effective amount of a neutrophil elastase inhibitor, in particular alvelestat or a pharmaceutically acceptable salt and/or solvate thereof to the subject.

The present invention also relates to methods for improving lung function in a subject referred to in this disclosure, in particular a subject with fibrosis associated with GVHD affecting the lungs, said method comprising administering an effective amount of a NE inhibitor, in particular alvelestat or a pharmaceutically acceptable salt and/or solvate thereof to the subject. In some embodiments the GVHD is chronic GVHD.

The present invention also relates to methods for preventing worsening of lung function in a subject referred to in this disclosure, in particular a subject with fibrosis associated with GVHD affecting the lungs said method comprising administering an effective amount of a NE inhibitor, in particular alvelestat or a pharmaceutically acceptable salt and/or solvate thereof to the subject. In some embodiments the GVHD is chronic GVHD.

The present invention also relates to methods for stabilising lung function in a subject referred to in this disclosure, in particular patients with fibrosis associated with GVHD affecting the lungs said method comprising administering an effective amount of a NE inhibitor, in particular alvelestat or a pharmaceutically acceptable salt and/or solvate thereof to the subject. In some embodiments the GVHD is chronic GVHD.

The present invention also relates to methods for preventing progression or worsening of fibrosis in a subject referred to in this disclosure, in particular a subject with GVHD, such as cGVHD. The present invention also relates to methods for stabilising fibrosis in a subject referred to in this disclosure, in particular a subject with GVHD, such as cGVHD. In some embodiments the GVHD is chronic GVHD.

The present invention also relates to methods for preventing progression of fibrosis in a subject referred to in this disclosure, in particular a subject with GVHD, such as cGVHD. The present invention also relates to methods for stabilising fibrosis in a subject referred to in this disclosure, in particular a subject with GVHD, such as cGVHD.

Also provided is alvelestat or a pharmaceutically acceptable salt and/or solvate thereof for use in treating or preventing fibrosis associated with graft rejection. In some embodiments the graft rejection is chronic or acute graft rejection. Also provided is alvelestat or a pharmaceutically acceptable salt thereof for use in treating or preventing fibrosis associated with lung transplant associated bronchiolitis obliterans syndrome. Also provided is alvelestat or a pharmaceutically acceptable salt thereof for use in treating or preventing fibrosis associated with GVHD.

Also provided is the use of alvelestat or a pharmaceutically acceptable salt and/or solvate thereof for the manufacture of a medicament for treating or preventing fibrosis associated with graft rejection. In some embodiments the graft rejection is chronic. Also provided is the use of alvelestat or a pharmaceutically acceptable salt thereof for the manufacture of a medicament for treating or preventing fibrosis associated with lung transplant associated bronchiolitis obliterans syndrome. Also provided is the use of alvelestat or a pharmaceutically acceptable salt thereof for the manufacture of a medicament for treating or preventing fibrosis associated with GVHD.

In general, any method of treating or preventing a disease described herein, comprising the administration of a product in an effective amount to a subject in need thereof, provides a corresponding embodiment to: that product for use in a method of treating or preventing the disease described herein. In addition, an embodiment is provided to: use of the product in the manufacture of a medicament for the treatment or prevention of the disease described herein.

### Methods

As described above, the present invention demonstrates that a subject with increased levels of biomarkers has elevated levels of activity, e.g. NE activity.

Thus, the present invention provides a method of identifying a subject in need of treatment with alvelestat or a pharmaceutically acceptable salt thereof, comprising determining the level of desmosine and isodesmosine in a sample from a subject, wherein a raised level of desmosine and isodesmosine relative to a baseline or a reference level identifies the subject in need of treatment.

A raised level of desmosine and isodesmosine relative to a baseline or a reference level equate to raised neutrophil elastase activity. The level of desmosine and isodesmosine can be determined by taking a sample, (e.g. a blood sample from a subject), and determining the level of the biomarker in a method (e.g. an ex vivo method).

Thus, the present invention further provides a method of determining neutrophil elastase activity, comprising evaluating the level of desmosine and isodesmosine in a sample from a subject, wherein a raised level of desmosine and isodesmosine relative to a baseline or a reference level indicate raised neutrophil elastase activity.

The present also invention provides a method of identifying a subject in need of treatment with alvelestat or a pharmaceutically acceptable salt thereof, comprising determining levels of PRO-C3 and/or PRO-C6 in a sample from a subject, wherein raised levels of PRO-C3 and/or PRO-C6 relative to a baseline or a reference level identifies the subject in need of treatment.

The present invention also provides a method of monitoring fibrotic activity in a patient undergoing treatment with a neutrophil elastase inhibitor, in particular alvelestat, the method comprising the steps of:
obtaining a sample from a subject,
measuring the level of a biomarker of collagen synthesis (e.g. PRO-C3) in the sample and measuring the level of a biomarker of collagen degradation (e.g. C3M) in the sample,
evaluating the ratio of the level of the biomarker of collagen synthesis (e.g. PRO-C3) to the level of the biomarker of collagen degradation (e.g. C3M) in a subject,
comparing the ratio against a baseline or a reference ratio,
wherein a decrease in the ratio relative to the baseline or the reference ratio indicates a decrease in fibrotic activity.

The present invention also provides a method of identifying a subject in need of treatment with a neutrophil elastase inhibitor, in particular alvelestat, wherein the subject has a fibrotic disease, the method comprising the steps of:

-obtaining a sample from a subject,
measuring the level of a biomarker of collagen synthesis (e.g. PRO-C3) in the sample and measuring the level of a biomarker of collagen degradation (e.g. C3M) in the sample,
evaluating the ratio of the level of the biomarker of collagen synthesis (e.g. PRO-C3) to the level of the biomarker of collagen degradation (e.g. C3M) in a subject,
comparing the ratio against a baseline or a reference ratio,
wherein an increase in the ratio relative to the baseline or the reference ratio identifies that the subject is in need of treatment with the neutrophil elastase inhibitor.

In some embodiments, the subject is diagnosed with BOS. In some embodiments, the BOS is GVHD BOS. In some embodiments, the subject has received a hematopoietic stem cell transplant.

In some embodiments, the levels of PRO-C3, PRO-C6, C3M, and/or C6M are measured by chromatography and/or mass spectrometry, for example isotopic dilution liquid chromatography tandem with mass spectrometry. In some embodiments, the sample from a subject is a plasma sample.

### Dosing

For the above mentioned therapeutic indications, the dose of the neutrophil inhibitor to be administered, in particular alvelestat or a pharmaceutically acceptable salt and/or solvate thereof, will depend on the disease being treated, the severity of the disease, the mode of administration, the age, weight and sex of the patient. Such factors may be determined by the attending physician. However, in general, satisfactory results are obtained when the compounds are administered to a human at a daily dosage of between 0.1 mg/kg to 100 mg/kg (measured as the active ingredient).

Suitably the daily dose is from 0.5 to 1000 mg per day, for example from 50 to 800 mg per day, in particular 50 to 600 mg per day, more particularly 120 mg to 550 mg, even more particularly 200 to 500 mg. For example the daily dose is about 240, 270, 300, 330, 360, 390, 420, 450 or 480 mg per day. The dose may be administered as a single dose or as a divided dose, for example wherein the total daily dose is divided in to two or more fractions, administered during the day. A dose may be administered daily, or multiple times a day (for example twice daily), or multiple times a week, or monthly, or multiple times a month.

In a particular embodiment alvelestat or a pharmaceutically acceptable salt and/or solvate thereof is administered twice a day (BID dosing). In a further embodiment alvelestat or a pharmaceutically acceptable salt and/or solvate thereof is administered twice a day, wherein each dose is equivalent to up to 240 mg of alvelestat free base, for example 60 mg twice a day, 90 mg twice a day, 120 mg twice a day, 150 mg twice a day, 180 mg twice a day, 210 mg twice a day, or 240 mg twice a day. In particular, 120 mg is administered twice a day or 240 mg is administered twice a day.

Compounds may be administered to an individual in accordance with an effective dosing regimen for a desired period of time or duration, such as at least one week, at least about one month, at least about 2 months, at least about 3 months, at least about 6 months, at least about 12 months, at least about 24 months, or longer. For example, the compound may be administered on a daily or intermittent schedule for the duration of the subject's life.

The dose of alvelestat or a pharmaceutically acceptable salt and/or solvate thereof may be administered according to a dosage escalation regime in all methods of the invention. This allows safe titration up to the standard daily dose of alvelestat, e.g. of 240 mg twice daily. For example, a dosage escalation regime up to a standard 240 mg twice daily dose of alvelestat according to the invention comprises administration of alvelestat or a pharmaceutically acceptable salt and/or solvate thereof at a dose of 60 mg of alvelestat twice daily for a first period of time, followed by 120 mg twice daily for a second period of time, followed by 180 mg twice daily for a third period of time, and 240 mg twice daily thereafter. The first, second and third periods may each be from 10-20 days, e.g. each about two weeks. In particular, alvelestat or a pharmaceutically acceptable salt and/or solvate thereof is administered at 60 mg twice daily for two weeks, followed by 120 mg twice daily for two weeks, followed by 180 mg twice daily for two weeks, and 240 mg twice daily thereafter. Doses are referred to as the equivalent amount of alvelestat free base.

### Compositions

The neutrophil inhibitor, in particular alvelestat or a pharmaceutically acceptable salt and/or solvate thereof, is administered to a subject in the form of a pharmaceutical composition.

Accordingly, the invention provides a method of treating or preventing any of the conditions described herein comprising administering a pharmaceutical composition comprising an effective amount of a neutrophil inhibitor, in particular alvelestat or a pharmaceutically acceptable salt and/or solvate thereof, and one or more pharmaceutically acceptable excipients, to a subject in need thereof.

Pharmaceutical compositions may be prepared with one or more pharmaceutically acceptable excipients which may be selected in accord with ordinary practice.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans. All compositions may optionally contain excipients such as those set forth in the Shesky et al, Handbook of Pharmaceutical Excipients, 8th edition, 2017. Excipients can include ascorbic acid and other antioxidants, chelating agents such as EDTA, carbohydrates such as dextrin, hydroxyalkylcellulose, hydroxyalkylmethylcellulose, stearic acid and the like.

Pharmaceutical compositions include those suitable for various administration routes, including oral administration. The compositions may be presented in unit dosage form and may be prepared by any of the methods well known in the art of pharmacy. Such methods include the step of bringing into association the active ingredient (e.g., a compound of the present disclosure or a pharmaceutical salt thereof) with one or more pharmaceutically acceptable excipients. The compositions may be prepared by uniformly and intimately bringing into association the active ingredient with liquid excipients or finely divided solid excipients or both, and then, if necessary, shaping the product. Techniques and formulations generally are found in Remington: The Science and Practice of Pharmacy, 22^{nd} Edition, 2012.

A preferred pharmaceutical composition is a solid dosage form, including a solid oral dosage form, such as a tablet. Tablets may contain excipients including glidants, fillers, binders and the like.

In effecting the methods described herein, the pharmaceutical compositions can be administered in any form and route which makes the compound bioavailable. Thus, the pharmaceutical compositions can be administered by a variety of routes, including oral and parenteral routes, more particularly by inhalation, subcutaneously, intramuscularly, intravenously, transdermally, intranasally, rectally, vaginally, occularly, topically, sublingually, and buccally, intraperitoneally, intravenously, intraarterially, transdermally, sublingually, intramuscularly, rectally, transbuccally, intranasally, intraadiposally, intrathecally and via local delivery for example by catheter or stent. Preferably, the pharmaceutical compositions are administered orally.

When used for oral use, tablets, troches, lozenges, aqueous or oil suspensions, dispersible powders or granules, emulsions, hard or soft capsules, syrups or elixirs may be prepared. Compositions described herein that are suitable for oral administration may be presented as discrete units (a unit dosage form) including but not limited to capsules, cachets or tablets each containing a predetermined amount of the active ingredient. Preferably, the pharmaceutical composition is a tablet.

Aqueous compositions may be prepared in sterile form, and when intended for delivery by other than oral administration generally may be isotonic.

The amount of active ingredient that may be combined with the inactive ingredients to produce a dosage form may vary depending upon the intended treatment subject and the particular mode of administration.

### Combination Therapy

In the present invention, the methods may further include the step of administering to the subject one or more additional therapeutic agents. The administration of the one or more additional therapeutic agents may occur prior to, concurrently with, or after the administration of the neutrophil inhibitor.

Additional therapeutic agents include immunosuppressive agents, anti-infective agents, anti-inflammatory agents, anti-fibrotic agents, and pain relievers.

In particular embodiments, the one or more additional therapeutic agent are immunosuppressive agents. For example, one, two, or preferably three immunosuppressive agents may be administered.

The immunosuppressive agents may, for example, be selected from the group consisting of corticosteroids (e.g. methylprednisolone, prednisone, prednisolone, budesonide, dexamethasone), janus kinase inhibitors (e.g. tofacitinib), calcineurin inhibitors (e.g. cyclosporine, tacrolimus), mTOR inhibitors (e.g. sirolimus, everolimus, temsirolimus), biologics (e.g. abatacept, adalimumab, anakinra, certolizumab, etanercept, golimumab, infliximab, ixekizumab, natalizumab, rituximab, secukinumab, tocilizumab, ustekinumab, vedolizumab), monoclonal antibodies (e.g. basiliximab, daclizumab), tyrosine kinase inhibitors (e.g. imatinib), thalidomide, pentostatin, azathioprine, mycophenolate and methotrexate.

In certain embodiments, the methods further include the step of administering to the subject a triple combination of immunosuppressive agents, for example tacrolimus, mycophenolate and a corticosteroid.

The one or more additional therapeutic agents may be anti-infective agents. Anti-infective agents include antibiotics, antifungals, anthelmintics, antimalarials, antiprotozoals, antituberculosis agents, and antivirals.

The one or more additional therapeutic agents may be anti-inflammatory agents. Anti-inflammatory agents include steroids, such as glucocorticoids. Anti-inflammatory agents may be selected from hydrocortisone, cortisone, prednisone, prednisolone, methylprednisone, methylprednisolone, dexamethasone, betamethasone, triamcinolone, fludrocortisone acetate, deoxycorticosterone acetate, aldosterone, and beclometasone.

The one or more additional therapeutic agents may be anti-fibrotic agents. Anti-fibrotic agents may be selected from nintedanib and pirfenidone.

The one or more additional therapeutic agents may be selected from the group of budesonide, beclomethasone dipropionate, cyclosporine, tacrolimus, sirolimus, mycophenolate mofetil, tilomisole, imuthiol, antithymocyte globulin, azathioprine, azodiacarbonide, bisindolyl maleimide VIII, brequinar, chlorambucil, CTLA4-Ig, cyclophosphamide, deoxyspergualin, leflunomide, mercaptopurine, 6-mercaptopurine, methotrexate, mizoribine, mizoribine monophosphate, muromonab CD3, mycophenolate mofetil, OKT3, rho (D) immune globin, vitamin D analogs, MC1288), daclizumab, infliximab, rituximab, tocilizumab alemtuzumab, methotrexate, antithymocyte globulin, denileukin diftitox, Campath-1H, keratinocyte growth factor, abatacept, remestemcel-L suberoylanilide hydroxamic acid, pentostatin, thalidomide, imatinib mesylate, cyclophosphamide, fludarabine, OKT3, melphalan, thiopeta, and lymphocyte immune globulin, anti-thymocyte, and globulin.

It is also understood that each of the agents administered individually or combined in a combination therapy or regimen may be administered at an initial dose that may then over time be reduced by a medical professional to reach a lower effective dose. For instance, in the combinations and regimens herein, systemic glucocorticosteroids (corticosteroids), such as prednisone and methyl prednisone may be administered to a human patient at a dose of from about 1-2 mg/kg/day. Initial daily doses for mTOR agents include sirolimus at 2-40 mg given once daily and everolimus at 0.25-1 mg given twice daily. Initial daily doses for calcineurin agents include tacrolimus at from about 0.025-0.2 mg/kg/day and cyclosporine at from about 2.5-9 mg/kg/day. Mycophenolate mofetil (CellCept®) may be administered at an initial daily dose of about 250-3,000 mg/day. Each of these agents may be administered in combination with a pharmaceutically effective amount of a Syk inhibitor as described herein following hematopoietic cell transplant. In different embodiments herein, agents useful in treating GVHD may be administered topically to a human in need of such treatment, such as in the form of a topical ointment or cream or in an eye drop formulation.

The present invention also provides methods for treating GVHD further including the step of administering light therapy (also known as extracorporeal photopheresis).

### EXAMPLES

Embodiments provided herein may be more fully understood by reference to the following examples. These examples are meant to be illustrative of methods provided herein, but are not in any way limiting. It will be apparent to those skilled in the art that various changes and modifications may be made. Such modifications are also intended to fall within the scope of the appended claims.

Alvelestat used in the following examples may be synthesised according to WO 2005/026123 A1 (Example 94, page 85).

From WO2021/053058 (herein incorporated by reference, and in particular see Examples 1-5) alvelestat:
is a potent and specific inhibitor of neutrophil elastase (NE);
shows a protective effect against GVHD;
can be used as a prophylaxis to the development of BOS in patients following lung transplantation;
can be used in the treatment for BOS in patients following lung transplantation; and
is being evaluated in a phase 1 Study of alvelestat in patients with bronchiolitis obliterans syndrome (BOS) after hematopoietic cell transplantation (HCT).

Example 1: a Phase 1b Study of Alvelestat, An Oral Neutrophil Elastase Inhibitor, In Patients with Bronchiolitis Obliterans Syndrome After Hematopoietic Cell Transplantation: Effect on elevated elastase and collagen turnover biomarkers

### Introduction

Bronchiolitis Obliterans Syndrome (BOS) is a rare but devastating complication of chronic graft-versus-host disease (GVHD) after allogeneic hematopoietic cell transplantation (HCT) and is associated with a high morbidity and mortality. There is a dearth of treatment options for BOS and new strategies are needed. Airway neutrophilia is a hallmark of BOS, even in the absence of infection, and neutrophil elastase (NE) is an enzyme that has been implicated in the pathogenesis of BOS. A phase 1b study of an oral NE inhibitor, alvelestat, in patients with BOS after HCT is being conducted. Biomarkers, including the elastin breakdown peptides desmosine (DES) and isodesmosine (IDES), and stimulated neutrophil elastase were used to assess direct effect on NE activity. Neo-epitope by-products of collagen type 3 and 6 synthesis (PRO-C3 and PRO-C6) are measured as biomarkers of fibrosis/tissue modelling. Neo-epitope by-products of collagen type 3 and 6 degradation (C3M and C6M) can also be measured as biomarkers of fibrosis/tissue modelling.

### Methods

Patients age ≥18 years with BOS and chronic GVHD after HCT were recruited to the National Cancer Institute protocol (NCT02669251). This phase 1 study had 2 parts: 8-week intra-patient dose escalation period, followed by a continuation period that allowed for up to 6 months of treatment. Alvelestat was given orally starting at 60 mg twice daily, increased every 2 weeks to 120 mg twice daily, 180 mg twice daily, and finally 240 mg twice daily.

Blood biomarkers for neutrophil elastase activity, desmosine, isodesmosine, PRO-C3 and PRO-C6, were collected at baseline (pre-treatment with alvelestat) and after every 2 weeks during alvelestat dose escalations i.e. after 60 mg bid, 120 mg bid, 180 mg bid and 240 mg bid.

DES and IDES levels in the plasma were measured by an isotope-dilution, liquid-chromatography/mass spectrometry method (Huang et al Thorax 2012; 67:502-508).[12] [13]

Biomarkers of novel epitopes (neoepitopes) related to collagen turnover and fibrosis (PRO-C6, PRO-C3, C3M, and C6M) were measured by competitive Enzyme Linked Immunosorbant Assay (ELISA) (Nordic Biosciences, Denmark).[1]

Ex-vivo zymosan-stimulated Neutrophil Elastase (NE) activity was measured by the following assay: zymosan was added to whole blood to induce degranulation of white blood cells including neutrophils, releasing an excess of free active NE into the plasma. The blood was centrifuged and plasma removed. The level of NE in the plasma sample was measured by the ProteaseTag® Active Neutrophil Elastase Immunoassay (ProAxsis Ltd, Belfast, Northern Ireland).

Results are presented as Mean and Standard Error Mean (SEM). Samples were taken at baseline and at the end of each dose-escalation stage.

### Results

7 patients were enrolled (3 men and 4 women). Median FEV1 after bronchodilator at time of enrolment was 44% predicted (range 38-74). All 7 patients were able to tolerate dose escalation of alvelestat up to the maximum dose 240 mg twice daily.

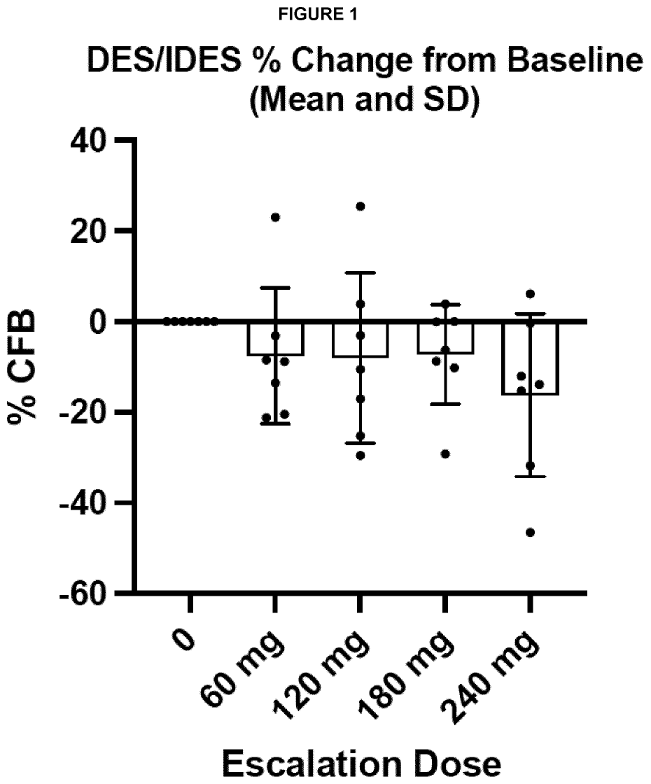

DES and IDES was elevated at baseline (mean 0.464 (SEM 0.0508) ng/ml, with 6 of 7 subjects above the Upper Limit of Normal (ULN, 0.280 ng/ml)). Levels progressively declined during the dose escalation period to 0.380 (SEM 0.0419) ng/ml by week 8, representing a mean within subject % change from baseline (CFB) of −16.2% (SEM 6.794, FIG. 1).

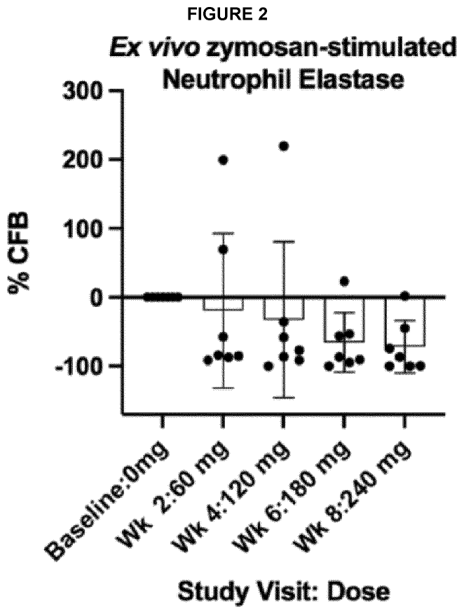

Ex vivo zymosan stimulated elastase activity also showed progressive decrease over the dose escalation period, with some subjects demonstrating 100% suppression (FIG. 2).

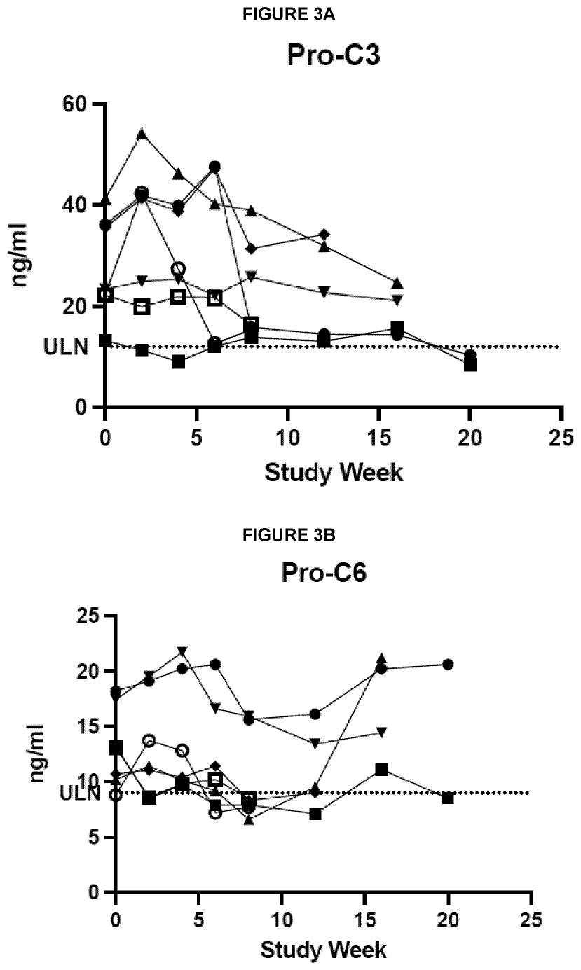

Collagen syntheses as measured by PRO-C3 and PRO-C6 was increased above ULN at baseline and declined with alvelestat treatment (FIGS. 3A and 3B).

There was consistency of a suppressive effect on biomarkers of elastase activity and collagen turnover in 6 of 7 treated patients, all of whom had improved or stable lung disease (defined as FEV1% predicted at end of treatment no worse than 10% below baseline level).

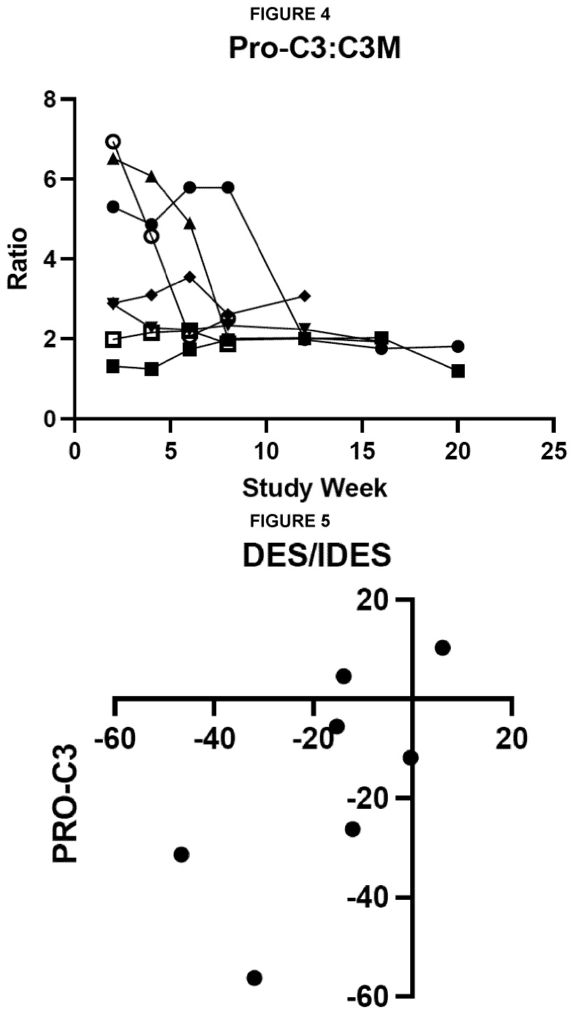

An indication of reduced fibrotic activity can be observed in FIG. 4 which shows a decrease in the ratio of PRO-C3 (a collagen synthesis biomarker) to C3M (a collagen synthesis biomarker) during the study.

These results also support a mechanistic link between inhibition of elastase by alvelestat and the anti-fibrotic effect. FIG. 5 shows the percent change from baseline (% CFB) for DES/IDES (elastin biomarkers) against PRO-C3 (collagen synthesis biomarker) in subjects. As seen from FIG. 5, there is positive correlation which is indicative of the mechanistic link.

### Conclusion

This is the first evidence of elevated elastase activity as detected by elastin breakdown in patients with cGVHD BOS.

Treatment with the selective NE inhibitor, alvelestat was associated with progressive reduction of plasma desmosine and idesmosine levels over 8 weeks with dose escalation and reduction stimulated neutrophil elastase activity. Treatment with the selective NE inhibitor, alvelestat was associated with progressive reduction of at least plasma PRO-C3 over 8 weeks with dose escalation.

Thus, inhibition of NE is suppressing elastase activity because the level of elastin breakdown biomarkers is decreasing. Inhibition of NE is also inhibiting fibrosis because the levels biomarkers associated with collagen synthesis are decreasing. Furthermore, the data also suggests a mechanistic link between NE inhibition by alvelestat and the reduction of fibrosis given the tandem suppression of elastin and collagen synthesis biomarkers. A way to observe the decrease in fibrosis can be from the ratio of relevant biomarkers, such as the level of a collagen synthesis biomarker to the level of a collagen degradation biomarker (indicative of fibrosis remodelling), e.g. PRO-C3:C3M.

The consistent suppression of elastase and of collagen synthesis biomarkers following alvelestat treatment is encouraging for its potential to impact progressive fibrosis, e.g. fibrosis associated with cGVHD BOS or lung fibrosis.

These data make plausible the treatment of fibrosis in a subject with an inhibitor of neutrophil elastase.

### Example 2: A 12-Week Study Treating Participants with Alvelestat or Placebo (NCT03636347)

As mentioned above, there is a link between the synthesis of collagen, a major component of the extracellular matrix (ECM), and fibrosis. Indeed, collagen degradation and formation (e.g. by ECM remodelling) are elevated in COPD compared with stable disease [Chest 2018; 154(4): 798-807], and ECM remodelling is a key element of fibrosis [Matrix Biol. (2018) 68-69, 122-149].

An aspect of NCT03636347 was to investigate the effect of alvelestat on collagen biomarkers in patients with PiZZ, null or rare variant phenotype/genotype alpha-1 anti-trypsin deficient lung disease. % Change from baseline in a number collagen degradation biomarkers were measured over a 12 week period in alvelestat and placebo arms.

### Methods

Inclusion criteria: patients (18 Years to 75 Years) with a confirmed diagnosis of alpha-1-anti-trypsin deficiency and a PiZZ, null or other rare geno/phenotype and serum anti-alpha1 antitrypsin levels of less than 11 uM; FEV1≥20% predicted; Computerised tomography (CT) scan evidence of emphysema; and non-smokers. Alvelestat was given orally at 120 mg twice daily, 240 mg twice daily, or dose escalation up to 240 mg twice daily, so there were three alvelestat treatment arms.

Collagen degradation biomarkers C1M (collagen Type 1), C6M (collagen Type 6), C4Ma3 (collagen Type 4) C3M (collagen Type 3) were collected at baseline (e.g. before treatment with alvelestat or placebo) and after 12 weeks following treatment with alvelestat or placebo.

Biomarkers related to C3M and C6M were measured by competitive Enzyme Linked Immunosorbant Assay (ELISA) (Nordic Biosciences, Denmark).[1] Biomarkers related to C1M and C4Ma3 were measured with Nordic Bioscience assays [Chest. 2017; 151(1): 47-59; and Lancet Respir Med. 2015; 3(6): 462-472]. C1M, C6M, C4Ma3 and C3M concentrations were measured in ng/mL.

Samples were taken at baseline (baseline is defined as last value prior to first dose of study drug) and at 12 weeks (i.e. value at week 12 or end of trial value) following the administration of alvelestat or placebo.

### Results and Discussion

Results are presented as the percentage change from baseline for each biomarker in alvelestat (the three treatment arms are represented in one data point) and placebo after 12 weeks, following ANCOVA analysis for the change from baseline (Least Square (LS) Means and LS Mean differences are taken from the model without interaction). SE=standard error. N=number of patients in study.

### Results:

TABLE 1

| | Change from baseline (%) | Change from baseline (%) |
|---|---|---|
| Biomarker | alvelestat at week 12 | placebo at week 12 |
| C1M | N = 32 | N = 22 |
| LS Means (SE) | −0.6 (49.42) | 17.9 (38.64) |
| C6M | N = 36 | N = 29 |
| LS Means (SE) | 1.1 (5.52) | 4.5 (5.98) |
| C4Ma3 | N = 17 | N = 11 |
| LS Means (SE) | −6.6 (8.96) | 1.9 (8.63) |
| C3M | N = 34 | N = 28 |
| LS Means (SE) | −1.7 (4.08) | 4.0 (4.26) |

From Table 1, patients administered alvelestat (the alvelestat arms) had a −0.6% change from baseline of collagen degradation biomarker C1M. By contrast patients administered placebo (placebo arm) had a 17.9% change from baseline of collagen degradation biomarker C1M. For C6M, the alvelestat arm had a 1.1% change from baseline and the placebo arm had a 4.5% change from baseline. For C4Ma3, the alvelestat arm had a-6.6% change from baseline and the placebo arm had a 1.9% change from baseline. For C3M, the alvelestat arm had a-1.7% change from baseline and the placebo arm had a 4.0% change from baseline. In general, the alvelestat arm showed less % change from baseline for the biomarkers in comparison to the placebo arm.

### Conclusion

This indicates that all four biomarkers showed numerically less turnover (e.g. ‘lower number of’ and ‘change’ values) with alvelestat than placebo. This supports a reduction in rate of turnover of ECM and collagen remodelling. ECM remodelling is a key element of fibrosis and through attenuation of this process alvelestat may impact fibrosis.

These data make plausible the treatment of fibrosis in a subject with an inhibitor of neutrophil elastase.

### REFERENCES

[1] Organ et al. Respiratory Research (2019) 20:148 (https://doi.org/10.1186/s12931-019-1118-7)
[2] Verleden S E, Vos R, Vanaudenaerde B M, Verleden G M, J Thorac Dis 2017; 9(8): 2650-2659
[3] Geert M. Verleden. The Journal of Heart and Lung Transplantation, Vol 38, No 5, 2019, pp 493-503
[4] Meyer K C, Raghu G, Verleden G M, Corris P A, Aurora P, Wilson K C, Brozek J, Glanville A R and the ISHLT/ATS/ERS BOS Task Force Committee; Eur Respir J 2014; 44:1479-1503
[5] Schlomchik W D, Nature Reviews Immunology, vol. 7, pages 340-352(2007).
[6] Tiberi S. et al. Eur. Respir. J. 2016; 47:333-336.
[7] Stevens T, Ekholm K, Granse M, Lindahl M, Kozma V, Jungar C, Ottosson T, Falk-Hakansson H, Churg A, Wright J L, Lal H, Sanfridson A. AZD9668: pharmacological characterization of a novel oral inhibitor of neutrophil elastase. The Journal of pharmacology and experimental therapeutics. 2011; 339(1): 313-20. Epub 2011 Jul. 28. doi: 10.1124/jpet. 111.182139. PubMed PMID: 21791628
[8] Groutas W C, Dou D, Alliston K R, Expert Opin Ther Pat. 2011 March; 21(3): 339-354
[9] Lee S J, Blood (2017) 129(1): 30-37
[10]Lee S J et al; Biol Blood Marrow Transplant 2002; 8(8): 444-52
[11] Nielsen N J et al. Am J Transl Res. 2013 Apr. 19; 5(3): 303-15
[12] Nedergaard A. et al. J Cachexia Sarcopenia Muscle (2013) 4:267-275.
[13] Huang et al. Thorax 2012; 67:502-508
[14] Albarbarawi O. et al. Bioanalysis. 2013 August; 5(16): 1991-2001 (doi: 10.4155/bio.13.164)

The invention also provides the following numbered embodiments.
1. A method for treating or preventing fibrosis, comprising administering an effective amount of alvelestat or a pharmaceutically acceptable salt and/or solvate thereof to a human subject in need thereof.
2. The method of embodiment 1, wherein the fibrosis is selected from the group consisting of arthrofibrosis, liver fibrosis, heart fibrosis, mediastinal fibrosis, retroperitoneal cavity fibrosis, bone marrow fibrosis, bridging fibrosis, nephrogenic systemic fibrosis, skin fibrosis, scleroderma and systemic sclerosis.
3. A method for treating or preventing fibrosis associated with a tissue, comprising administering an effective amount of alvelestat or a pharmaceutically acceptable salt and/or solvate thereof to a human subject in need thereof.
4. The method of embodiment 3, wherein the tissue comprises one or more tissue selected from the group consisting of lung tissue, liver tissue, brain tissue, heart tissue, gastrointestinal tissue, and skin tissue.
5. The method of embodiment 3 or 4, wherein the tissue is lung tissue.
6. The method of any of embodiments 3 to 5, wherein the tissue is associated with graft versus host disease (GVHD).
7. A method for treating or preventing fibrosis associated with a disease, comprising administering an effective amount of alvelestat or a pharmaceutically acceptable salt and/or solvate thereof to a human subject in need thereof.
8. The method of embodiment 7, wherein the disease is one or more selected from the group consisting of graft rejection, graft versus host disease (GVHD), chronic lung allograft dysfunction (CLAD), Lung Transplant associated Bronchiolitis Obliterans Syndrome (LT-BOS), Lung Transplant associated Restrictive Allograft Syndrome (LT-RAS), bronchiolitis obliterans syndrome (BOS), graft versus host disease associated bronchiolitis obliterans syndrome (GVHD BOS), graft versus host disease associated restrictive chronic lung function decline (GVHD R-CLFD), liver disease, heart disease, cirrhosis, fibrothorax, radiation-induced lung injury, glial scar, arterial stiffness, kidney disease, Crohn's disease, Dupuytren's contracture, keloid disorder, adhesive capsulitis, rheumatoid arthritis, ulcerative colitis, systemic lupus erythematosus, and hypertension.
9. A method for treating or preventing fibrosis associated with graft rejection, comprising administering an effective amount of alvelestat or a pharmaceutically acceptable salt and/or solvate thereof to a human subject in need thereof.
10. A method for treating or preventing graft rejection, comprising administering an effective amount of alvelestat or a pharmaceutically acceptable salt and/or solvate thereof to a human subject in need thereof, wherein the treatment comprises reducing fibrosis in the subject.
11. The method of any of embodiments 8-10, wherein the graft comprises one or more organs selected from the group consisting of skin, kidney, heart, liver, lung and pancreas.
12. The method of embodiment 11, wherein the graft comprises a lung.
13. The method of any preceding embodiment, wherein the subject has, or is at risk of having, lung transplant associated bronchiolitis obliterans syndrome.
14. The method of any preceding embodiment, wherein the graft rejection is chronic graft rejection.
15. A method for treating or preventing fibrosis associated with lung transplant associated bronchiolitis obliterans syndrome, comprising administering an effective amount of alvelestat or a pharmaceutically acceptable salt and/or solvate thereof to a human subject in need thereof.
16. A method for treating or preventing lung transplant associated bronchiolitis obliterans syndrome, comprising administering an effective amount of alvelestat or a pharmaceutically acceptable salt and/or solvate thereof to a human subject in need thereof wherein the treatment comprises reducing fibrosis in the subject.
17. A method for treating or preventing fibrosis associated with graft versus host disease (GVHD), comprising administering an effective amount of alvelestat or a pharmaceutically acceptable salt and/or solvate thereof in a human subject in need thereof.
18. A method for treating or preventing graft versus host disease (GVHD), comprising administering an effective amount of alvelestat or a pharmaceutically acceptable salt and/or solvate thereof in a human subject in need thereof wherein the treatment comprises reducing fibrosis in the subject.
19. The method of any of embodiments 8 or 17-18, wherein the GVHD is chronic GVHD (cGVHD).
20. The method of embodiment 19, wherein the GVHD is acute GVHD (aGVHD).
21. The method of any of embodiments 8 or 17-20, wherein the GVHD manifests after bone marrow transplantation.
22. The method of any one of embodiments 8 or 17-21, wherein the GVHD manifests after hematopoietic stem cell transplantation.
23. The method of any one of embodiments 8 or 17-22, wherein the GVHD is characterised by damage to one or more selected from the group consisting of the eyes, joints, fascia, genital organ, lung, liver, skin, or gastrointestinal tract (e.g. mouth, oesophagus).
24. The method of any one of embodiments 8 or 17-23, wherein the GVHD is characterised by damage to one or more selected from the group consisting of the lung, liver, skin, or gastrointestinal tract.
25. The method of any one of embodiments 8 or 17-24, where in the subject has moderate or severe cGVHD.
26. The method of any one of embodiments 8 or 17-25, wherein the subject has, or is at risk of having, bronchiolitis obliterans syndrome.
27. A method for treating or preventing fibrosis associated with bronchiolitis obliterans syndrome (BOS) and GVHD, comprising administering an effective amount of alvelestat or a pharmaceutically acceptable salt and/or solvate thereof to a human subject in need thereof.
28. A method for treating or preventing bronchiolitis obliterans syndrome (BOS) associated with GVHD, comprising administering an effective amount of alvelestat or a pharmaceutically acceptable salt and/or solvate thereof to a human subject in need thereof, wherein the treatment comprises reducing fibrosis in the subject.
29. The method of any of embodiments 8 or 27-28, wherein the BOS is associated with hematopoietic stem cell transplant.
30. The method of embodiment 29, where the hematopoietic stem cell transplant is an allogeneic hematopoietic cell transplant.
31. The method of any of embodiments 8 or 27-28, wherein the BOS is associated with bone marrow transplant.
32. The method of any preceding embodiment, wherein the subject has neutrophilia.
33. The method of embodiment 32, wherein the neutrophilia is airway neutrophilia.
34. The method of any preceding embodiment, wherein alvelestat or a pharmaceutically acceptable salt and/or solvate thereof is administered prior to transplantation into the subject.
35. The method of any one of embodiments 1 to 33, wherein alvelestat or a pharmaceutically acceptable salt and/or solvate thereof is administered after transplantation into the subject.
36. The method of any preceding embodiment, wherein the treatment or prevention comprises inhibiting neutrophil elastase.
37. The method of any preceding embodiment, wherein the treatment, prevention or reduction of fibrosis comprises reducing the level of one or more biomarkers.
38. The method of any preceding embodiment, wherein the subject has elevated levels of one or more biomarkers.
39. The method of any preceding embodiment, wherein the treatment, prevention, or reduction of fibrosis comprises reducing the level of one or more biomarkers over 12 weeks.
40. The method of any of embodiments 37-39, wherein the biomarker is a collagen biomarker.
41. The method of embodiment 40, wherein the collagen biomarker is a type III collagen biomarker and/or a type IV biomarker.
42. The method of embodiment 40 or 41, wherein the collagen biomarker is an N-terminal propeptide of type III collagen (PRO-C3) and/or an N-terminal propeptide of type VI collagen (PRO-C6).
43. The method of embodiment 40 or 41, wherein the collagen biomarker is the N-terminal propeptide of type III collagen (PRO-C3).
44. The method of any of embodiments 37-39, wherein the biomarker is an elastin biomarker.
45. The method of embodiment 44, wherein the elastin biomarker is selected from desmosine (DES) and isodesmosine (IDES).
46. The method of any preceding embodiment, wherein the treatment or prevention comprises improving or preventing worsening of the FEV1% predicted in the subject.
47. The method of any preceding embodiment, wherein the treatment or prevention comprises improving or preventing worsening of the BOS grade of the subject.
48. The method of any preceding embodiment wherein the treatment of cGVHD comprises improving the cGVHD severity score in a subject.
49. The method of any preceding embodiment, wherein the treatment of cGVHD comprises improving the Lee cGVHD Symptom Scale in a subject, in particular the Lee cGVHD Symptom Scale lung score in a subject with cGVHD affecting a lung.
50. The method of any preceding embodiment comprising improving lung function in a subject.
51. The method of any preceding embodiment, comprising preventing worsening of lung function in a subject.
52. The method of any preceding embodiment, comprising preventing progression or worsening of disease in a subject.
53. The method of any preceding embodiment, wherein alvelestat is in the form of the free base.
54. The method of any preceding embodiment, wherein alvelestat is in the form of alvelestat tosylate.
55. The method of any preceding embodiment, comprising administering alvelestat or a pharmaceutically acceptable salt and/or solvate thereof twice daily.
56. The method of any preceding embodiment, comprising administering alvelestat or a pharmaceutically acceptable salt and/or solvate thereof at a dose of alvelestat of up to 240 mg twice daily.
57. The method of any preceding embodiment, comprising administering alvelestat or a pharmaceutically acceptable salt and/or solvate thereof at a dose of alvelestat of 60 mg, 120 mg, 180 mg or 240 mg twice daily.
58. The method of any preceding embodiment, comprising administering alvelestat or a pharmaceutically acceptable salt and/or solvate thereof at a dose of alvelestat of 240 mg twice daily.
59. The method of any preceding embodiment, comprising administering alvelestat or a pharmaceutically acceptable salt and/or solvate thereof at a dose of alvelestat of 60 mg twice daily for a first period of time, followed by 120 mg twice daily for a second period of time, followed by 180 mg twice daily for a third period of time, and 240 mg twice daily thereafter.
60. The method of any preceding embodiment, comprising administering alvelestat or a pharmaceutically acceptable salt and/or solvate thereof at a dose of alvelestat of 60 mg twice daily for two weeks, followed by 120 mg twice daily for two weeks, followed by 180 mg twice daily for two weeks, and 240 mg twice daily thereafter.
61. The method of any preceding embodiment, comprising administering alvelestat or a pharmaceutically acceptable salt and/or solvate thereof by oral administration.
62. The method of any preceding embodiment, further comprising administering to the subject one or more immunosuppressive agents.
63. A method of identifying a human subject in need of treatment with alvelestat or a pharmaceutically acceptable salt thereof, comprising determining the level of desmosine and isodesmosine in a sample from the subject, wherein a raised level of desmosine and isodesmosine relative to a baseline identifies the subject in need of treatment.
64. The method of embodiment 63, wherein the raised level of desmosine and isodesmosine relative to a baseline equate to raised neutrophil elastase activity.
65. A method of determining neutrophil elastase activity, comprising evaluating the level of desmosine and isodesmosine in a sample from a human subject, wherein a raised level of desmosine and isodesmosine relative to a baseline indicate raised neutrophil elastase activity.
66. The method of any of embodiments 63-65, wherein the subject is diagnosed with GVHD.
67. The method of embodiment 66, wherein the GVHD is GVHD BOS.
68. The method of embodiment 66 or 67, wherein the subject has received a hematopoietic stem cell transplant.
69. The method of any of embodiments 63-68, wherein the level of the biomarker, such as desmosine and isodesmosine, are measured by chromatography and/or mass spectrometry, for example isotopic dilution liquid chromatography tandem with mass spectrometry.
70. The method of any of embodiments 63-69, wherein the sample from the subject is a blood sample, for example a plasma sample.

1. A method for treating or preventing fibrosis, comprising administering an effective amount of alvelestat or a pharmaceutically acceptable salt and/or solvate thereof to a human subject in need thereof.

2. The method of claim 1, wherein the fibrosis is selected from the group consisting of liver fibrosis, arthrofibrosis, heart fibrosis, mediastinal fibrosis, retroperitoneal cavity fibrosis, bone marrow fibrosis, bridging fibrosis, hepatic fibrosis, nephrogenic systemic fibrosis, skin fibrosis, scleroderma and systemic sclerosis, for example liver fibrosis.

3. A method for treating or preventing fibrosis associated with a tissue, comprising administering an effective amount of alvelestat or a pharmaceutically acceptable salt and/or solvate thereof to a human subject in need thereof.

4. The method of claim 3, wherein the tissue comprises one or more tissue selected from the group consisting of liver tissue, lung tissue, brain tissue, heart tissue, gastrointestinal tissue, and skin tissue, for example wherein the tissue is lung tissue.

5. The method of claim 3 or 4, wherein the tissue is associated with graft versus host disease (GVHD).

6. A method for treating or preventing fibrosis associated with a disease, comprising administering an effective amount of alvelestat or a pharmaceutically acceptable salt and/or solvate thereof to a human subject in need thereof.

7. The method of claim 6, wherein the disease is one or more selected from the group consisting of graft rejection, graft versus host disease (GVHD), chronic lung allograft dysfunction (CLAD), Lung Transplant associated Bronchiolitis Obliterans Syndrome (LT-BOS), Lung Transplant associated Restrictive Allograft Syndrome (LT-RAS), bronchiolitis obliterans syndrome (BOS), graft versus host disease associated bronchiolitis obliterans syndrome (GVHD BOS), graft versus host disease associated restrictive chronic lung function decline (GVHD R-CLFD), liver disease, heart disease, cirrhosis, fibrothorax, radiation-induced lung injury, glial scar, arterial stiffness, kidney disease, Crohn's disease, Dupuytren's contracture, keloid disorder, adhesive capsulitis, rheumatoid arthritis, ulcerative colitis, systemic lupus erythematosus, hypertension, non-alcoholic fatty liver disease (NAFLD), non-alcoholic steatosis, and non-alcoholic steatohepatitis (NASH); for example, wherein disease is one or more selected from non-alcoholic fatty liver disease (NAFLD), non-alcoholic steatosis, non-alcoholic steatohepatitis (NASH), and cirrhosis.

8. A method for treating or preventing fibrosis associated with graft rejection, comprising administering an effective amount of alvelestat or a pharmaceutically acceptable salt and/or solvate thereof to a human subject in need thereof.

9. A method for treating or preventing graft rejection, comprising administering an effective amount of alvelestat or a pharmaceutically acceptable salt and/or solvate thereof to a human subject in need thereof, wherein the treatment comprises reducing fibrosis in the subject.

10. The method of any of claims 7-9, wherein the graft comprises one or more organs selected from the group consisting of skin, kidney, heart, liver, lung and pancreas, for example wherein the graft comprises a lung.

11. The method of any preceding claim, wherein:
(i) the subject has, or is at risk of having, lung transplant associated bronchiolitis obliterans syndrome; and/or
(ii) the graft rejection is chronic graft rejection.

12. A method for treating or preventing fibrosis associated with lung transplant associated bronchiolitis obliterans syndrome, comprising administering an effective amount of alvelestat or a pharmaceutically acceptable salt and/or solvate thereof to a human subject in need thereof.

13. A method for treating or preventing lung transplant associated bronchiolitis obliterans syndrome, comprising administering an effective amount of alvelestat or a pharmaceutically acceptable salt and/or solvate thereof to a human subject in need thereof wherein the treatment comprises reducing fibrosis in the subject.

14. A method for treating or preventing fibrosis associated with graft versus host disease (GVHD), comprising administering an effective amount of alvelestat or a pharmaceutically acceptable salt and/or solvate thereof in a human subject in need thereof.

15. A method for treating or preventing graft versus host disease (GVHD), comprising administering an effective amount of alvelestat or a pharmaceutically acceptable salt and/or solvate thereof in a human subject in need thereof wherein the treatment comprises reducing fibrosis in the subject.

16. The method of any of claim 7 or 14-15, wherein:
(i) the GVHD is chronic GVHD (cGVHD), or wherein the GVHD is acute GVHD (aGVHD); and/or
(ii) the GVHD manifests after bone marrow transplantation; and/or
(iii) the GVHD manifests after hematopoietic stem cell transplantation; and/or
(iv) the GVHD is characterised by damage to one or more selected from the group consisting of the eyes, joints, fascia, genital organ, lung, liver, skin, or gastrointestinal tract (e.g. mouth, oesophagus); and/or
(v) the GVHD is characterised by damage to one or more selected from the group consisting of the lung, liver, skin, or gastrointestinal tract; and/or
(vi) the subject has moderate or severe cGVHD; and/or
(vii) the subject has, or is at risk of having, bronchiolitis obliterans syndrome.

17. A method for treating or preventing fibrosis associated with bronchiolitis obliterans syndrome (BOS) and GVHD, comprising administering an effective amount of alvelestat or a pharmaceutically acceptable salt and/or solvate thereof to a human subject in need thereof.

18. A method for treating or preventing bronchiolitis obliterans syndrome (BOS) associated with GVHD, comprising administering an effective amount of alvelestat or a pharmaceutically acceptable salt and/or solvate thereof to a human subject in need thereof, wherein the treatment comprises reducing fibrosis in the subject.

19. The method of any of claim 7 or 17-18, wherein:
(i) the BOS is associated with hematopoietic stem cell transplant, for example where the hematopoietic stem cell transplant is an allogeneic hematopoietic cell transplant; or
(ii) the BOS is associated with bone marrow transplant.

20. The method of any preceding claim, wherein:
(i) the subject has neutrophilia, for example wherein the neutrophilia is airway neutrophilia; and/or
(ii) alvelestat or a pharmaceutically acceptable salt and/or solvate thereof is administered prior to or after transplantation into the subject; and/or
(iii) the treatment or prevention comprises inhibiting neutrophil elastase.

21. The method of any preceding claim, wherein:
(i) the treatment, prevention or reduction of fibrosis comprises reducing the level of one or more biomarkers; and/or
(ii) the subject has elevated levels of one or more biomarkers; and/or
(iii) the treatment, prevention, or reduction of fibrosis comprises reducing the level of one or more biomarkers over 12 weeks.

22. The method of claim 21, wherein:
(i) the biomarker is a collagen biomarker,
for example wherein the collagen biomarker is a type III collagen biomarker and/or a type IV biomarker, optionally:
wherein the collagen biomarker is an N-terminal propeptide of type III collagen (PRO-C3) and/or an N-terminal propeptide of type VI collagen (PRO-C6), for example
wherein the collagen biomarker is the N-terminal propeptide of type III collagen (PRO-C3); or
(ii) the biomarker is an elastin biomarker,
for example wherein the elastin biomarker is selected from desmosine (DES) and isodesmosine (IDES).

23. The method of any preceding claim, wherein:
(i) the treatment or prevention comprises improving or preventing worsening of the FEV1% predicted in the subject; and/or
(ii) the treatment or prevention comprises improving or preventing worsening of the BOS grade of the subject; and/or
(iii) the treatment of cGVHD comprises improving the cGVHD severity score in a subject; and/or
(iv) the treatment of cGVHD comprises improving the Lee cGVHD Symptom Scale in a subject, in particular the Lee cGVHD Symptom Scale lung score in a subject with cGVHD affecting a lung.

24. The method of any preceding claim:
(i) comprising improving lung function in a subject; and/or
(ii) comprising preventing worsening of lung function in a subject; and/or
(iii) comprising preventing progression or worsening of disease in a subject.

25. The method of any preceding claim, wherein:
(i) alvelestat is in the form of the free base and/or
(ii) alvelestat is in the form of alvelestat tosylate.

26. The method of any preceding claim:
(i) comprising administering alvelestat or a pharmaceutically acceptable salt and/or solvate thereof twice daily; and/or
(ii) comprising administering alvelestat or a pharmaceutically acceptable salt and/or solvate thereof at a dose of alvelestat of up to 240 mg twice daily; and/or
(iii) comprising administering alvelestat or a pharmaceutically acceptable salt and/or solvate thereof at a dose of alvelestat of 60 mg, 120 mg, 180 mg or 240 mg twice daily; and/or
(iv) comprising administering alvelestat or a pharmaceutically acceptable salt and/or solvate thereof at a dose of alvelestat of 240 mg twice daily; and/or
(v) comprising administering alvelestat or a pharmaceutically acceptable salt and/or solvate thereof at a dose of alvelestat of 60 mg twice daily for a first period of time, followed by 120 mg twice daily for a second period of time, followed by 180 mg twice daily for a third period of time, and 240 mg twice daily thereafter; and/or
(vi) comprising administering alvelestat or a pharmaceutically acceptable salt and/or solvate thereof at a dose of alvelestat of 60 mg twice daily for two weeks, followed by 120 mg twice daily for two weeks, followed by 180 mg twice daily for two weeks, and 240 mg twice daily thereafter; and/or
(vii) comprising administering alvelestat or a pharmaceutically acceptable salt and/or solvate thereof by oral administration; and/or
(viii) further comprising administering to the subject one or more immunosuppressive agents.

27. A method of identifying a human subject in need of treatment with alvelestat or a pharmaceutically acceptable salt thereof, comprising determining the level of desmosine and isodesmosine in a sample from the subject, wherein a raised level of desmosine and isodesmosine relative to a baseline identifies the subject in need of treatment.

28. The method of claim 27, wherein the raised level of desmosine and isodesmosine relative to a baseline equate to raised neutrophil elastase activity.

29. A method of determining neutrophil elastase activity, comprising evaluating the level of desmosine and isodesmosine in a sample from a human subject, wherein a raised level of desmosine and isodesmosine relative to a baseline indicate raised neutrophil elastase activity.

30. The method of any of claims 27-29, wherein:
(i) the subject is diagnosed with GVHD, optionally: wherein the GVHD is GVHD BOS, and/or the subject has received a hematopoietic stem cell transplant; and/or
(ii) the level of the biomarker, such as desmosine and isodesmosine, are measured by chromatography and/or mass spectrometry, for example isotopic dilution liquid chromatography tandem with mass spectrometry; and/or
(vi) wherein the sample from the subject is a blood sample, for example a plasma sample.
